(19) United States
(12) Patent Application Publication
(10) Pub. No.: US 2016/0272673 A1
(43) Pub. Date: Sep. 22, 2016

(54) ISOLATION AND PURIFICATION OF DVD-IGS

(71) Applicant: ABBVIE INC., North Chicago, IL (US)

(72) Inventors: Heidi Althouse, Worcester, MA (US); Shilpa Ananthakrishnan, Worcester, MA (US); Germano Coppola, Shrewsbury, MA (US); Scott T. Ennis, South Grafton, MA (US); Robert K. Hickman, Worcester, MA (US); Chen Wang, Shrewsbury, MA (US); Joe Yakamavich, Worcester, MA (US)

(21) Appl. No.: 15/035,088

(22) PCT Filed: Nov. 7, 2014
(86) PCT No.: PCT/US14/64636

§ 371 Date: May 6, 2016

Related U.S. Application Data
(60) Provisional application No. 61/901,242, filed on Nov. 7, 2013.
(60) Provisional application No. 61/901,214, filed on Nov. 7, 2013.
(60) Provisional application No. 61/901,228, filed on Nov. 7, 2013.
(60) Provisional application No. 61/901,183, filed on Nov. 7, 2013.

(51) Int. Cl. C07K 1/16; A61K 39/395; C07K 16/00

(52) U.S. Cl. CPC C07K 1/165; C07K 16/00; A61K 39/395

(57) ABSTRACT

Chromatographic methods for isolating and purifying DVD-lgs™ from a sample, wherein the purified DVD-lgs™ have reduced host cell proteins, aggregates, and viruses compared to the sample.

### STATEMENT REGARDING FEDERAL FUNDING

Embodiments of the present invention were not conceived or developed with Federal sponsorship or funding.

### BACKGROUND OF THE INVENTION

Dual variable domain immunoglobulins, or DVD-Igs™, are a new class of protein therapeutics which have distinct molecular and separation characteristics from monoclonal antibodies. This may present great challenges for purification to meet pharmaceutical product requirements. Purification processes for pharmaceutical grade DVD-Ig™ produced by fermentation culture may involve four basic steps. These steps include (1) harvest/clarification—separation of host cells from the fermentation culture; (2) capture—separation of DVD-Ig™ from the majority of components in the clarified harvest; (3) fine purification—removal of residual product- and process-related impurities such as host cell contaminants and aggregates; and (4) formulation—place the DVD-Ig™ into an appropriate carrier for maximum stability and shelf life.

Among the four steps, the fine purification may comprise one or more chromatographic steps such as ion exchange chromatography (IEX) and hydrophobic interaction chromatography (HIC). Although these methods have been used to purify proteins, such as antibodies, they sometimes present technical difficulties in the separation of product-related impurities (e.g. aggregated and fragmented species) and/or process-related contaminants (e.g. viruses and host cell proteins) at desired product recovery (e.g. ≧85%). Moreover, the process throughput with these methods is often limited, with resin loading capacity typically below 100 g/L. As a result, a large size of column has to be used for large scale manufacturing which significantly increases the operating cost.

To enhance the fine purification process performance new generation chromatography resins are continuously being developed by vendor companies. One class of resins recently introduced to the field is the multimodal (MM) chromatography resins which utilize ion-exchange, hydrophobic and/or other modes of interactions. Among them, mixed mode resins containing cation exchange functionality have been developed for direct capture of target proteins from clarified harvest and for bind-elute fine purification of proteins. Although these methods can improve protein selectivity, and in some cases increase binding capacities for certain protein systems, the impact on the overall process throughput is incremental compared to traditional methods. Thus, there is an existing need for protein purification methods that utilize the high capacity and selectivity properties of cation exchanger-based mixed mode (CEX-MM) resins but avoid the manufacturing constraints defined by bind and elute technology. The present invention addresses these purification needs for DVD-Igs™.

### SUMMARY OF THE INVENTION

The present invention is directed to methods for isolating and purifying DVD-Igs™ from a sample using various chromatographic separation methodologies. In certain aspects, the invention is directed to methods of DVD-Ig™ purification employing affinity chromatography, preferably Protein A chromatography. In certain aspects, the methods herein employ an affinity chromatography step and one or more additional chromatography and/or filtration steps. The chromatography steps can include one or more steps of ion exchange chromatography and/or hydrophobic interaction chromatography. In particular aspects, cation-exchanger-based mixed mode (CEX-MM) flow-through chromatography are employed. In certain aspects, the present invention provides a method for removing product- and process-related impurities at substantially higher throughput than conventional bind-elute methods and with high product recovery. In other aspects, the present invention can be used with other chromatographic and/or filtration techniques to achieve desired protein product quality. In certain aspects, the present invention provides methods for purifying a DVD-Ig product from a sample mixture, wherein the DVD-Ig product contains a decreased number of viral particles or decreased viral activity in comparison to the sample mixture.

One embodiment of the present invention is directed toward a method of purifying a DVD-Ig™ from a sample such that the resulting DVD-Ig™ composition is substantially free of product- and process-related impurities including potential viruses. In one aspect, the sample comprises a cell line harvest wherein the cell line is employed to produce specific DVD-Igs™ of the present invention.

One method of the present invention involves clarifying a cell culture sample comprising a DVD-Ig™ of interest using a centrifuge and/or depth filtration to obtain a clarified harvest. The clarified sample is then subjected to capture chromatography.

In one embodiment, the capture chromatography step comprises subjecting the primary recovery sample to a column comprising a suitable affinity chromatographic support. Non-limiting examples of such chromatographic supports include Protein A resin, Protein G resin, affinity supports comprising the antigen against which the DVD-Ig™ of interest was raised, and affinity supports comprising an Fc binding protein. In certain embodiments, Protein A resins are employed for the affinity purification of DVD-Igs™. In certain aspects, the affinity chromatography sample is collected and further subjected to subsequent chromatographic steps such as ion exchange and hydrophobic interactive chromatography.

In one aspect, a Protein A column is equilibrated with a suitable buffer prior to sample loading. A non-limiting example of a suitable buffer is a Tris/NaCl buffer, pH around 7.2. Following this equilibration, the sample can be loaded onto the column. Following the loading of the column, the column can be washed one or multiple times using, e.g., the equilibrating buffer. Other washes including washes employing different buffers can be used before eluting the column. The Protein A column can then be eluted using an appropriate elution buffer. A non-limiting example of a suitable elution buffer is an acetic acid/NaCl buffer, pH around 3.5. The eluate can be monitored using techniques well known to those skilled in the art. For example, the absorbance at OD₂₈₀ can be followed. The eluated fraction(s) of interest can then be prepared for further processing.

In one embodiment, the affinity chromatography sample is collected, incubated at low pH to inactivate enveloped virus if present, followed by pH adjustment to neutral or more basic conditions for further polishing. In an embodiment, the pH adjusted Protein A eluate is then filtered through a depth filter followed by a Q membrane adsorber in flow-through mode. A non-limiting example of the Q membrane is Mustang Q membrane adsorber (Pall Life Sciences), Sartobind Q or STIC membrane adsorber (Sartorius), or a Qyuspeed Q membrane adsorber (Asahi Kasei).

In one embodiment, the Q membrane flow-through eluate pool is collected and then further flowed through a CEX-MM column to reduce aggregates, HCP and/or other impurities including protein fragments. In another embodiment, the Q membrane and the CEX-MM column can be run in tandem in a continuous manner. In one aspect, the CEX-MM column is packed with Capto MMC™ ImpRes resin. In another aspect, the CEX-MM column is packed with Nuvia™ cPrime resin. In yet another aspect, the CEX-MM column is packed with Capto MMC™ or Toyopearl MX Trp-650M resin.

In an embodiment, the Q membrane flow-through pool is further adjusted to pH about 5.0-7.5 and conductivity adjusted to 3-20 mS/cm for the CEX-MM polishing step. In another embodiment, the column can be equilibrated using a suitable buffer prior to loading the sample (the Q flow-through pool) onto the mixed mode column. A non-limiting example of a suitable buffer is a Tris/Acetate buffer with a pH of about 5-7. Following equilibration, the column is loaded with the Q flow-through pool. Following loading, the column can be washed one or multiple times with a suitable buffer. A non-limiting example of a suitable buffer is the equilibration buffer. Flow-through collection can commence, e.g., as the absorbance (OD₂₈₀) rises above about 0.2 AU.

In another embodiment, the sequences of the Q membrane and the CEX-MM steps can be reversed. In yet another embodiment, the Q membrane flow-through pool can be processed through an AEX-MM resin such as Capto™ Adhere ImpRes in the flow-through mode before processing through a CEX-MM column. In a further embodiment the capture chromatography sample is subjected to mixed mode flow through chromatography without the need for further purification.

In one embodiment, the present invention provides a method of purifying a DVD-Ig™ comprising the steps of (a) adjusting the sample with acid or a base to a pH of about 1-4 pH units lower than the pI of the protein of interest and adjusting the sample conductivity to about 2-20 mS/cm; (b) equilibrating a column packed with CEX-MM resin with a buffer having a similar pH and conductivity as the adjusted sample; (c) applying the adjusted sample to the CEX-MM column at resin loading level of about 200 to 1200 g/L; and (d) washing the column with equilibration buffer and collecting the product flow-through and wash pool. In other embodiments, the CEX-MM column can be flowed at a residence time of 1 to 6 minutes. In yet another embodiment, the CEX-MM resin is in contact with the protein mixture in a batch adsorption mode and the supernatant containing the desired protein is collected as the final product.

In certain embodiments, a suitable CEX-MM resin stationary phase contains anionic and hydrophobic groups. Non-limiting examples of such a resin include Capto MMC™, Capto MMC™ ImpRes (GE Healthcare), Nuvia™ cPrime (BioRad), and ToyoPearl MX Trp-650M (Tosoh Bioscience).

In one embodiment, the invention is directed to methods of DVD-Ig™ purification employing two or more chromatographic steps consisting of capture chromatography such as Protein A chromatography, a CEX-MM flow-through chromatography, and one or two additional chromatography steps for polishing. The additional chromatography steps can include ion exchange, hydrophobic interaction, and/or a mixed mode chromatography.

In one embodiment, the ion exchange step is an anion exchange chromatography. Examples of the anion exchangers include but are not limited to Mustang Q (Pall), Sartobind Q, Sartobind STIC (Sartorius) or Qyuspeed Q (Asahi Kaise) membrane adsorbers, Q Sepharose Fast Flow, Capto Q (GE Healthcare), Nuvia Q (BioRad), Poros HQ (Life Technology) resins. In another embodiment, the additional mixed mode chromatography step is an anion exchange based mixed mode (AEX-MM) chromatography. Non-limiting examples of the AEX-MM resins are Capto™ Adhere and Capto™ Adhere ImpRes resins (GE).

In one embodiment, the affinity chromatography eluate is prepared for anion exchange polishing by adjusting the pH and ionic strength of the sample buffer. For example, the affinity eluate can be adjusted to a pH of about 5.0 to about 8.5 and the conductivity adjusted to 2-15 mS/cm and then diluted to about 5-12 g/L prior to loading on the Q membrane.

In one embodiment, an anion ion exchange step follows Protein A affinity chromatography. A non-limiting example of an anion exchange step is a Q Sepharose™ column, a QyuSpeed™ D (QSD) membrane absorber, or a Sartobind Q membrane absorber. In an embodiment, the anion exchange chromatography procedure operates in flow through mode wherein the DVD-Ig™ of interest does not interact or bind to the anion exchange resin (or solid phase). In this embodiment, many impurities such as viruses, host cell proteins, DNA, aggregates, and, where applicable, affinity matrix protein do interact with and bind to the anion exchange resin.

In some embodiments, the affinity chromatography eluate is prepared for anion ion exchange by adjusting the pH and ionic strength of the sample buffer. For example, the affinity eluate can be adjusted to a pH of about 6.0 to about 8.5 using a 1 M Tris buffer. Prior to loading the sample (the affinity eluate) onto the ion exchange column, the column can be equilibrated using a suitable buffer. A non-limiting example of a suitable buffer is a Tris/NaCl buffer with a pH of about 6.0 to about 8.5. Following equilibration, the column can be loaded with the affinity eluate. Following loading, the column can be washed one or multiple times with a suitable buffer. A non-limiting example of a suitable buffer is the equilibration buffer itself. Flow-through collection can commence, e.g., as the absorbance (OD₂₈₀) rises above about 0.2 AU.

In particular embodiments, the eluate obtained following mixed mode flow-through or a combination of the embodiments described in this application can be subjected to a small virus retentive filtration followed by final ultrafiltration and diafiltration processing to achieve the targeted drug substance formulations. In certain embodiments, there is an overall reduction in the total number of viral particles in a DVD-Ig preparation compared to an eluate sample mixture. In specific embodiments, the overall reduction in the total number of viral particles in the DVD-Ig preparation is greater than a 3 log reduction value (LRV).

In certain embodiments, the small virus retentive filtration utilizes a nanofilter. In particular embodiments, the nanofilter comprises a material selected from polyestersulfone (PES), polyvinylidene fluoride (PVDF), and cellulose. Non-limiting examples of nanofilters include Zeta Plus VR™, Virosart™ CPV, Virosart™ HC, Virosart™ HF, Viresolve™ Pro, Ultipor® VF DV20, Planova™ 20N, and Planova™ BioEx.

In certain embodiments, the conductivity of the sample mixture subjected to viral filtration is about 2 to about 12 S/mmS/cm. In certain embodiments, the concentration of the DVD-Ig in the sample mixture is about 2 to about 10 g/L. In other embodiments, the pH of the sample mixture is about 5.0 to about 8.2.

In some embodiments, the pressure applied to the first end of the sample mixture loaded onto a nanofilter is about 14 to about 42 psi. In an embodiment, the flux through the nanofilter is about 0 to about 550 LMH. In an embodiment, the flux decay of the nanofilter is about 0 to about 100%. In an embodiment, the throughput of the nanofilter is about 0 to about 5 kg/m2. In an embodiment, the total yield of the DVD-Ig preparation is about 22 to about 100%.

In particular embodiments, the DVD-Ig in the sample mixture and/or DVD-Ig preparation has a retention time on a hydrophobic interaction chromatography (HIC) column of about 13 to about 22.5 min. In specific embodiments, the DVD-Ig in the sample mixture and/or DVD-Ig preparation has a greater average retention time than a monoclonal antibody or antigen binding fragment thereof, and optionally, a greater average retention time than the monoclonal antibody or antigen binding fragment thereof comprising at least one antigen binding domain of the DVD-Ig.

In certain embodiments, the DVD-Ig in the sample mixture and/or DVD-Ig preparation has a HIC elution profile half-height peak width of about 0.8 to about 2.7 min. In other embodiments, the DVD-Ig in the sample mixture and/or DVD-Ig preparation has a greater HIC elution profile half-height peak width than a monoclonal antibody or antigen binding fragment thereof, and optionally, a greater average retention time than the monoclonal antibody or antigen binding fragment thereof comprising at least one antigen binding domain of the DVD-Ig.

In an embodiment, the purity of the DVD-Ig™ of interest in the resultant sample product can be analyzed using methods well known to those skilled in the art, e.g., size-exclusion chromatography, Poros™ A HPLC Assay, HCP ELISA, Protein A ELISA, and western blot analysis.

In yet another embodiment, the invention is directed to one or more pharmaceutical compositions comprising an isolated DVD-Ig. In another aspect, the compositions further comprise one or more pharmaceutical agents.

### BRIEF DESCRIPTIONS OF THE DRAWINGS

FIG. 1 depicts DVD1 aggregates clearance by Capto MMC™ flow-through.

FIG. 2a depicts DVD1 aggregates clearance by Capto MMC™ ImpRes flow-through as a function of resin loading.

FIG. 2b depicts DVD1 aggregates clearance by Capto MMC™ ImpRes flow-through as a function of operating conditions.

FIG. 3 depicts DVD1 product pool aggregate levels as a function of Capto MMC™ ImpRes resin loading at pH 7 and 15 mS/cm.

FIG. 4a depicts cumulative aggregate levels for DVD1 as a function of Nuvia™ cPrime resin loading.

FIG. 4b depicts cumulative aggregate levels for DVD1 as a function of cumulative yield at pH 7 and 15 mS/cm buffer condition.

FIG. 5a depicts DVD2 flow-through pool aggregate levels as a function of Capto MMC™ ImpRes resin loading.

FIG. 5b depicts DVD2 flow-through pool aggregate levels as a function of yield under different operating conditions.

FIG. 6a depicts DVD2 flow-through pool aggregate levels as a function of Nuvia™ cPrime resin loading.

FIG. 6b depicts DVD2 flow-through pool aggregate levels as a function of yield under different operating conditions.

FIG. 7 depicts EA1 aggregate reductions and HCP clearance by QSD membrane absorber.

FIG. 8 depicts EA5 aggregate clearance by QSD membrane absorber.

FIG. 9 depicts EA6 aggregate and HCP clearance by QSD membrane absorber.

FIG. 10 depicts EA7 aggregate clearance by QSD membrane absorber.

FIG. 11 depicts EA7 HCP clearance.

FIG. 12 depicts aggregate reduction comparison for DVD-Igs.

FIG. 13 depicts the elution and various regeneration conditions during Phenyl HP bind-elute processing for DVD1.

FIG. 14 depicts a representative flow through chromatogram for DVD1.

FIG. 15 depicts a representative flow through chromatogram for DVD2.

FIG. 16 depicts the DVD2 Phenyl HP flow through process using 150 mM sodium citrate buffer.

FIG. 17 depicts representative SEC chromatograms for DVD2 Phenyl Load and FTW samples.

FIG. 18 depicts the chromatographic profile for DVD3 utilizing 200 mM sodium citrate, showing the relative location of the fragments, monomer and aggregates.

FIG. 19a depicts flux vs. throughput during DVD-1 viral filtration at pH 8, 2.3 g/L concentration.

FIG. 19b depicts flux vs. throughput during DVD-1 viral filtration at pH 5, 2.2 g/L concentration.

FIG. 19c depicts flux vs. throughput during DVD-1 viral filtration at pH 8, 9.8 g/L concentration.

FIG. 19d depicts flux vs. throughput during DVD-1 viral filtration at pH 5, 9.6 g/L concentration.

FIG. 20a depicts flux decay during DVD-1 viral filtration at pH 8, 2.3 g/L concentration.

FIG. 20b depicts flux decay during DVD-1 viral filtration at pH 5, 2.2 g/L concentration.

FIG. 20c flux decay during DVD-1 viral filtration at pH 8, 9.8 g/L concentration.

FIG. 20d depicts flux decay during DVD-1 viral filtration at pH 5, 9.6 g/L concentration.

FIG. 21a depicts flux vs. throughput during DVD-2 viral filtration at pH 6.5, 3 g/L concentration.

FIG. 21b depicts flux vs. throughput during DVD-2 viral filtration at pH 5, 3 g/L concentration.

FIG. 22a depicts flux decay during DVD-2 viral filtration at pH 6.5, 3 g/L concentration.

FIG. 22b depicts flux decay during DVD-2 viral filtration at pH 5, 3 g/L concentration.

FIG. 23a depicts flux vs. throughput during DVD-3 viral filtration at pH 6.8, 8 g/L concentration.

FIG. 23b depicts flux vs. throughput during DVD-3 viral filtration at pH 5, 8 g/L concentration.

FIG. 24a depicts flux decay during DVD-3 viral filtration at pH 6.8, 8 g/L concentration.

FIG. 24b depicts flux decay during DVD-3 viral filtration at pH 5, 8 g/L concentration.

FIG. 25 depicts the HIC retention times for DVD-Igs vs. mAbs.

FIG. 26 depicts the HIC elution profile half-height peak width for DVD-Igs vs. mAbs.

### DETAILED DESCRIPTION OF THE INVENTION

The present invention is directed to chromatographic and filtration methods for purifying DVD-Igs™ from a sample containing process- and product-related impurities including HCP, DNA, fragments, aggregates, and viruses. One aspect of the invention is directed to viral reduction of samples generated in the various steps of DVD-Ig™ purification. In a particular aspect, methods herein employ a viral filtration step. In another aspect, the viral filtration step may be preceded and/or followed by one or more chromatography steps. In a particular aspect, methods herein employ a cation exchanger-based mixed mode (CEX-MM) flow-through chromatography to reduce levels of aggregates, fragments, and/or HCPs in a sample containing DVD-Igs™ of interest. The CEX-MM purification can be used with other chromatographic methods such as Protein A, anion exchange (AEX) chromatography, hydrophobic interaction chromatography (HIC), and/or AEX-based mixed mode chromatography steps along with filtration steps to achieve efficient purification of DVD-Igs™. Further, the present invention is directed toward pharmaceutical compositions comprising one or more DVD-Ig™ purified by a method described herein.

For clarity and not by way of limitation, this detailed description is divided into the following sub-portions:

1. Definitions;

2. DVD-Ig™ Generation;

3. DVD-Ig™ Production;

4. DVD-Ig™ Purification;

5. Methods of Assaying Sample Purity;

6. Further Modifications; and

7. Pharmaceutical Compositions.

### 1. DEFINITIONS

In order that the present invention may be more readily understood, certain terms are first defined.

The term “binding protein” as used in this section refers to an intact binding protein or an antigen binding fragment thereof. Non-limiting examples of binding proteins include, for example, DVD-Igs™, or antigen binding fragments thereof.

The term dual variable domain immunoglobulin, sometimes referred herein as a DVD-Ig™ or DVD, is a binding protein comprising a polypeptide chain, wherein said polypeptide chain comprises VD1-(X1)n-VD2-C-(X2)n, wherein VD1 is a first variable domain, VD2 is a second variable domain, C is a constant domain, X1 represents an amino acid or polypeptide, X2 represents an Fc region, and n is 0 or 1. These immunoglobulins are described in U.S. Pat. No. 7,612,181, the entire teaching of which is incorporated by reference herein. In some embodiments, the VD1 and VD2 in the binding protein are heavy chain variable domains. In some embodiments the heavy chain variable domain is selected from the group consisting of a murine heavy chain variable domain, a human heavy chain variable domain, a CDR grafted heavy chain variable domain, and a humanized heavy chain variable domain. In some embodiments the VD1 and VD2 are capable of binding the same antigen. In other embodiment VD1 and VD2 are capable of binding different antigens. Preferably, C is a heavy chain constant domain. More preferably, X1 is a linker with the proviso that X1 is not CH1

An “isolated binding protein” includes a binding protein that is substantially free of other binding proteins having different antigenic specificities (e.g., an isolated binding protein that specifically binds a target antigen is substantially free of binding proteins that specifically bind antigens other than the target antigen). An isolated binding protein that specifically binds a target antigen may bind the same target antigen from other species. Moreover, an isolated binding protein may be substantially free of other cellular material and/or chemicals.

The phrase “recombinant host cell” (or simply “host cell”) includes a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “about”, as used herein, is intended to refer to ranges of approximately 10-20% greater than or less than the referenced value. In certain circumstances, one of skill in the art will recognize that, due to the nature of the referenced value, the term “about” can mean more or less than a 10-20% deviation from that value.

The phrase “viral reduction/inactivation”, as used herein, is intended to refer to a decrease in the number of viral particles in a particular sample (“reduction”), as well as a decrease in the activity, for example, but not limited to, the infectivity or ability to replicate, of viral particles in a particular sample (“inactivation”). Such decreases in the number and/or activity of viral particles can be on the order of about 1% to about 99%, preferably of about 20% to about 99%, more preferably of about 30% to about 99%, more preferably of about 40% to about 99%, even more preferably of about 50% to about 99%, even more preferably of about 60% to about 99%, yet more preferably of about 70% to about 99%, yet more preferably of about 80% to 99%, and yet more preferably of about 90% to about 99%. In certain non-limiting embodiments, the amount of virus, if any, in the purified binding protein product is less than the ID₅₀ (the amount of virus that will infect 50 percent of a target population) for that virus, preferably at least 10-fold less than the ID₅₀ for that virus, more preferably at least 100-fold less than the ID₅₀ for that virus, and still more preferably at least 1000-fold less than the ID₅₀ for that virus.

The tern “aggregates” used herein means agglomeration or oligomerization of two or more individual molecules, including but not limiting to, protein dimers, trimers, tetramers, oligomers and other high molecular weight species. Protein aggregates can be soluble or insoluble.

The term “fragments” used herein refers to any truncated protein species from the target molecule due to dissociation of peptide chain, enzymatic and/or chemical modifications

The term “host cell proteins” (HCPs), as used herein, is intended to refer to non-target protein-related, proteinaous impurities derived from host cells.

### 2. BINDING PROTEIN GENERATION

The term “binding protein” as used in this section refers to an intact DVD-Ig.

A binding protein preferably can be a human, a chimeric, or a humanized DVD-Ig™ Chimeric or humanized binding proteins of the present disclosure can be prepared based on the sequence of a non-human binding protein prepared as described above. DNA encoding the heavy and light chain immunoglobulins can be obtained from the non-human hybridoma of interest and engineered to contain non-murine (e.g., human) immunoglobulin sequences using standard molecular biology techniques. For example, to create a chimeric binding protein, murine variable regions can be linked to human constant regions using methods known in the art (see e.g., U.S. Pat. No. 4,816,567 to Cabilly et al.). To create a humanized binding protein, murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

In yet another embodiment of the invention, binding proteins can be altered wherein the constant region of the binding protein is modified to reduce at least one constant region-mediated biological effector function relative to an unmodified binding protein. To modify a binding protein of the invention such that it exhibits reduced binding to the Fc receptor, the immunoglobulin constant region segment of the binding protein can be mutated at particular regions necessary for Fc receptor (FcR) interactions (see, e.g., Canfield and Morrison (1991) J. Exp. Med. 173:1483-1491; and Lund et al. (1991) J. of Immunol. 147:2657-2662, the entire teachings of which are incorporated herein). Reduction in FcR binding ability of the binding protein may also reduce other effector functions which rely on FcR interactions, such as opsonization and phagocytosis and antigen-dependent cellular cytotoxicity.

### 3. BINDING PROTEIN PRODUCTION

To express a binding protein of the invention, DNAs encoding partial or full-length light and heavy chains are inserted into one or more expression vector such that the genes are operatively linked to transcriptional and translational control sequences. (See, e.g., U.S. Pat. No. 6,914,128, the entire teaching of which is incorporated herein by reference.) In this context, the term “operatively linked” is intended to mean that a binding protein gene is ligated into a vector such that transcriptional and translational control sequences within the vector serve their intended function of regulating the transcription and translation of the binding protein gene. The expression vector and expression control sequences are chosen to be compatible with the expression host cell used. The binding protein light chain gene and the binding protein heavy chain gene can be inserted into a separate vector or, more typically, both genes are inserted into the same expression vector. The binding protein genes are inserted into an expression vector by standard methods (e.g., ligation of complementary restriction sites on the binding protein gene fragment and vector, or blunt end ligation if no restriction sites are present). Prior to insertion of the binding protein light or heavy chain sequences, the expression vector may already carry binding protein constant region sequences. Additionally or alternatively, the recombinant expression vector can encode a signal peptide that facilitates secretion of the binding protein chain from a host cell. The binding protein chain gene can be cloned into the vector such that the signal peptide is linked in-frame to the amino terminus of the binding protein chain gene. The signal peptide can be an immunoglobulin signal peptide or a heterologous signal peptide (i.e., a signal peptide from a non-immunoglobulin protein).

A binding protein of the invention can be prepared by recombinant expression of DVD-Ig™ light and heavy chain genes in a host cell. To express a binding protein recombinantly, a host cell is transfected with one or more recombinant expression vectors carrying DNA fragments encoding the immunoglobulin light and heavy chains of the binding protein such that the light and heavy chains are expressed in the host cell and secreted into the medium in which the host cells are cultured, from which medium the binding protein can be recovered. Standard recombinant DNA methodologies are used to obtain binding protein heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel et al. (eds.) Current Protocols in Molecular Biology, Greene Publishing Associates, (1989) and in U.S. Pat. No. 4,816,397 & 6,914, 128, the entire teachings of which are incorporated herein.

Suitable host cells for clonuing or expressing the DNA in the vectors herein are prokaryote, yeast, or higher eukaryotic cells. Suitable mammalian host cells for expressing the recombinant binding protein of the invention include Chinese Hamster Ovary (CHO cells) (including dhfr-CHO cells, described in Urlaub and Chasin, (1980) PNAS USA 77:42164220, used with a DHFR selectable marker, e.g., as described in Kaufman and Sharp (1982) Mol. Biol. 159:601-621, the entire teachings of which are incorporated herein by reference), NSO myeloma cells, COS cells and SP2 cells. When recombinant expression vectors encoding binding protein genes are introduced into mammalian host cells, the binding proteins are produced by culturing the host cells for a period of time sufficient to allow for expression of the binding proteins in the host cells or secretion of the binding proteins into the culture medium in which the host cells are grown.

Host cells are transformed with the above-described expression or cloning vectors for binding protein production and cultured in conventional nutrient media modified as appropriate for inducing promoters, selecting transformants, or amplifying the genes encoding the desired sequences.

When using recombinant techniques, the binding protein can be produced intracellularly, in the periplasmic space, or directly secreted into the medium. Prior to the process of the invention, procedures for purification of binding proteins from cell debris initially depend on the site of expression of the binding protein. Some binding proteins can be secreted directly from the cell into the surrounding growth media; others are made intracellularly. Where the binding protein is secreted, supernatants from such expression systems are generally first concentrated using a commercially available protein concentration filter, e.g., an Amicon™ or Millipore Pellicon™ ultrafiltration unit. Where the binding protein is secreted into the medium, the recombinant host cells can also be separated from the cell culture medium, e.g., by tangential flow filtration. Binding proteins can be further recovered from the culture medium using the binding protein purification methods of the invention.

### 4. BINDING PROTEIN PURIFICATION

### 4.1 Binding Protein Purification Generally

The invention provides high-throughput chromatographic and filtration methods for producing a purified (or “HCP-, aggregate-, fragment- or virus-reduced”) DVD-Ig™ preparation from a mixture comprising a DVD-Ig™ and at least one HCP, aggregate, or virus. The purification process of the invention begins at the separation step when the DVD-Ig™ has been produced using methods described above and conventional methods in the art. Table 1 summarizes one embodiment of a purification scheme. Variations of this scheme, including, but not limited to, variations where the Protein A affinity chromatography step is omitted or the order of the non-affinity chromatography steps is reversed, are envisaged and are within the scope of this invention.

TABLE 1
Purification steps with their associated purpose

| Purification step | Purpose |
|---|---|
| Primary recovery | Clarification of sample matrix |
| Affinity chromatography | Binding protein capture, host cell protein |
| | and associated impurity reduction |
| Low pH inactivation | Inactivate viruses |
| Anion exchange | Removing host cell protein, DNA, |
| chromatography | and virus (if present) |
| Mixed mode | Reducing aggregates, fragments, HCPs, |
| chromatography | DNA, virus, leached Protein A |
| Hydrophobic interaction | Reducing aggregates, fragments, HCPs |
| chromatography | |
| Viral filtration | Removal of viruses, if present |
| Final ultrafiltration/ | Concentrate and formulate proteins |
| diafiltration | |

Once a clarified solution or mixture comprising the binding protein has been obtained, separation of the binding protein from the other proteins produced by the cell, such as HCPs, is performed using a combination of different purification techniques, including ion exchange separation step(s) and hydrophobic interaction separation step(s). The separation steps separate mixtures of proteins on the basis of their charge, degree of hydrophobicity, or size. In one aspect of the invention, separation is performed using chromatography, including cationic, anionic, and hydrophobic interaction. Several different chromatography resins are available for each of these techniques, allowing accurate tailoring of the purification scheme to the particular protein involved. The essence of each of the separation methods is that proteins can be caused either to traverse at different rates down a column, achieving a physical separation that increases as they pass further down the column, or to adhere selectively to the separation medium, being then differentially eluted by different solvents. In some cases, the binding protein is separated from impurities when the impurities specifically adhere to the column and the binding protein does not, i.e., the binding protein is present in the flow through.

### 4.7 Exemplary Purification Strategies

The initial steps of the purification methods of the present invention involve the first phase of clarification and primary recovery of DVD-Ig™ from a sample. In addition, the primary recovery process can also be a point at which to reduce or inactivate viruses that can be present in the sample. For example, any one or more of a variety of methods of viral reduction/inactivation can be used during the primary recovery phase of purification including heat inactivation (pasteurization), pH inactivation, solvent/detergent treatment, UV and X-ray irradiation and the addition of certain chemical inactivating agents such as 13-propiolactoneor e.g., copper phenanthroline as in U.S. Pat. No. 4,534,972, the entire teaching of which is incorporated herein by reference.

In certain embodiments, primary recovery can proceed by sequentially employing pH reduction/treatment, centrifugation, and filtration steps to remove cells and cell debris (including HCPs) from the production bioreactor harvest. For example, but not by way of limitation, a culture comprising DVD-Ig, media, and cells can be subjected to pH-mediated virus reduction/inactivation using an acid pH of about 3.5 (for an acidic pH of 5) for approximately 1 hour. The pH reduction can be facilitated using known acid preparations such as citric acid, e.g., 3 M citric acid. However, the pH of the sample mixture may be lowered by any suitable acid including, but not limited to, citric acid, acetic acid, caprylic acid, or other suitable acids. The choice of pH level largely depends on the stability profile of the binding protein product and buffer components. It is known that the quality of the target binding protein during low pH virus reduction/inactivation is affected by pH and the duration of the low pH incubation. In certain embodiments the duration of the low pH incubation will be from 0.5 hr to 2 hr, preferably 0.5 hr to 1.5 hr, and more preferably the duration will be 1 hr. Virus reduction/inactivation is dependent on these same parameters in addition to protein concentration, which may limit reduction/inactivation at high concentrations. Thus, the proper parameters of protein concentration, pH, and duration of reduction/inactivation can be selected to achieve the desired level of viral reduction/inactivation. Exposure to acid pH reduces, if not completely eliminates, pH sensitive viral contaminants and precipitates some media/cell contaminants. Following this viral reduction/inactivation step, the pH is adjusted to about 4.9 or 5.0 using a base such as sodium hydroxide, e.g., 3 M sodium hydroxide, for about twenty to about forty minutes. This adjustment can occur at around 20° C.

In those embodiments where viral reduction/inactivation is employed, the sample mixture can be adjusted, as needed, for further purification steps. For example, following low pH viral reduction/inactivation the pH of the sample mixture is typically adjusted to a more neutral pH, e.g., from about 4.5 to about 8.5, prior to continuing the purification process. Additionally, the mixture may be flushed with water for injection (WFI) to obtain a desired conductivity.

In certain embodiments, the primary recovery will include one or more centrifugation steps to further clarify the sample matrix and thereby aid in purifying the binding protein. In certain embodiments, the pH adjusted culture is then centrifuged at approximately 7000×g to approximately 12,750×g. In the context of large scale purification, such centrifugation can occur on-line with a flow rate set to achieve, for example, but not by way of limitation, a turbidity level of 150 NTU in the resulting supernatant. Such supernatant can then be collected for further purification.

In certain embodiments, the primary recovery will include the use of a filter train comprising one or more depth filtration steps to further clarify the sample matrix and thereby aid in purifying the DVD-Igs of the present invention. In certain embodiments, the filter train comprises around twelve 16-inch Cuno™ model 30/60ZA depth filters (3M Corp.) and around three round filter housings fitted with three 30-inch 0.45/0.2 μm Sartopore™ 2 filter cartridges (Sartorius). Depth filters contain filtration media having a graded density. Such graded density allows larger particles to be trapped near the surface of the filter while smaller particles penetrate the larger open areas at the surface of the filter, only to be trapped in the smaller openings nearer to the center of the filter. In certain embodiments the depth filtration step can be a delipid depth filtration step. Although certain embodiments employ depth filtration steps only during the primary recovery phase, other embodiments employ depth filters, including delipid depth filters, during one or more additional phases of purification. Non-limiting examples of depth filters that can be used in the context of the instant invention include the Millistak® X0HC, F0HC, C0HC, D0HC, A1HC, B1HC (Millipore), and Cuno™ model 30/60ZA depth filters (3M Corp.). A 0.2 μm filter is typically used after the depth filters to further remove fine particles. A non-limiting example of 0.2 μm filter is Sartopore™ 2 bilayer 0.45/0.2 μm filter cartridges. In certain embodiments, the resulting sample supernatant is then passed through a filter train comprising multiple depth filters. The clarified supernatant is collected in a vessel such as a pre-sterilized harvest vessel and held at approximately 2-12° C. This temperature is then adjusted to approximately 20° C. prior to the capture chromatography step or steps outlined below. It should be noted that one skilled in the art may vary the conditions recited above and still be within the scope of the present invention.

In certain embodiments, primary recovery will be followed by affinity chromatography using Protein A resin. There are several commercial sources for Protein A resin. One suitable resin is MabSelect™ or MabSelect SuRe™ from GE Healthcare, or ProSep Ultra Plus from EMD Millipore. A non-limiting example of a suitable column packed with MabSelect SuRe™ is a column about 1.0 cm diameter about 21.6 cm long (−17 mL bed volume). This size column can be used for bench scale. This can be compared with other columns used for scale ups. For example, a 20 cm×21 cm column whose bed volume is about 6.6 L can be used for commercial production. Regardless of the column, the column can be packed using a suitable resin such as MabSelect SuRe™.

In certain embodiments it will be advantageous to identify the dynamic binding capacity (DBC) of the Protein A resin in order to tailor the purification to the particular binding protein of interest. For example, but not by way of limitation, the DBC of a MabSelect™ column can be determined either by a single flow rate load or dual-flow load strategy. The single flow rate load can be evaluated at a velocity of about 300 cm/hr throughout the entire loading period. The dual-flow rate load strategy can be determined by loading the column up to about 35 mg protein/mL resin at a linear velocity of about 300 cm/hr, then reducing the linear velocity by half to allow longer residence time for the last portion of the load.

In certain aspects, the Protein A column can be equilibrated with a suitable buffer prior to sample loading. A non-limiting example of a suitable buffer is a Tris/NaCl buffer, pH of about 6 to 8, preferably about 7.2. A specific non-limiting example of suitable equilibration condition is 25 mM Tris, 100 mM NaCl, pH 7.2. Following this equilibration, the sample can be loaded onto the column. Following the loading of the column, the column can be washed one or multiple times using, e.g., the equilibrating buffer. Other washes including washes employing different buffers can be employed prior to eluting the column. For example, the column can be washed using one or more column volumes of 20 mM citric acid/sodium citrate, 0.5 M NaCl at pH of about 6.0. This wash can optionally be followed by one or more washes using the equilibrating buffer. The Protein A column can then be eluted using an appropriate elution buffer. A non-limiting example of a suitable elution buffer is an acetic acid buffer, pH around 3.5. Suitable conditions are, e.g., 0.1 M acetic acid, pH of about 3.5. The eluate can be monitored using techniques well known to those skilled in the art. For example, the absorbance at OD₂₈₀ can be followed. Column eluate can be collected starting with an initial deflection of about 0.5 AU to a reading of about 0.5 AU at the trailing edge of the elution peak. The elution fraction(s) of interest can then be prepared for further processing. For example, the collected sample can be titrated to a pH of about 5.0 using Tris (e.g., 1.0 M) at a pH of about 10. Optionally, this titrated sample can be filtered and further processed.

In certain embodiments, following the Protein A capture is a low pH viral inactivation step. In one embodiment, the eluate is adjusted to a pH of between about 3 and 4, and preferably at a pH of about 3.5, using a suitable acid including, but not limited to, citric acid, acetic acid, phosphoric acid. In certain embodiments the duration of the low pH incubation is from 0.5 hr to 2 hr, preferably 0.5 hr to 1.5 hr, and more preferably the duration will be 1 hr. Virus inactivation is dependent on these same parameters in addition to protein concentration, which may limit inactivation at high concentrations. Thus, the proper parameters of protein concentration, pH, and duration of inactivation can be selected to achieve the desired level of viral inactivation.

In certain embodiments, the inactivated Protein A eluate can then be adjusted to a pH of about 5 to 9 using a basic solution such as Tris or trolamine, and conductivity of 2-15 mS/cm using water or a suitable buffer, and filtered through a depth filter such as X0HC or CE50 to remove any particles or turbidity formed during this process. The filtrate is then flow-through an anion exchange step employing an anion exchange membrane adsorber. Non-limiting examples include Sartobind Q, Sartobind STIC (Sartorius), Mustang Q (Pall), QyuSpeed™ D (QSD, Ashi Kasei), and ChromaSorb (EMD Millipore). The anion exchange step may also be combined with a mixed mode chromatography process performed with resins having an anion exchange function and a hydrophobic interaction function. Examples of such mixed mode resins include but not limited to Capto™ Adhere and Capto™ Adhere ImpRes (GE Healthcare). The AEX membrane or the AEX-MM column is equilibrated with a wash buffer such as 20 mM Tris, pH 8.5. The membrane is challenged with the feed at a loading level of 1-3 kg/L, while the AEX-MM column is loaded with feed at a loading level of 200-300 g/L After loading the membrane or the column is flushed with 1-10 membrane/column volumes of equilibration buffer and the flow through and wash fractions collected and measured for protein concentrations by UV₂₈₀.

A skilled artisan may vary the conditions but still be within the scope of the present invention. The flow-through comprising the DVD-Ig™ can be monitored using a UV spectrophotometer at OD₂₈₀. This anion exchange step reduces product- and process-related impurities such as aggregates, nucleic acids like DNA, and host cell proteins. The separation occurs due to the fact that the DVD-Ig™ of interest do not substantially interact with nor bind to the anion exchanger, but many impurities do interact with and bind to the charged solid phase. The anion exchange can be performed at about 12-25° C.

In certain embodiments, the instant invention provides methods for producing a HCP-reduced binding protein preparation from a mixture comprising a binding protein and at least one HCP by subjecting the mixture to at least one ion exchange separation step such that an eluate comprising the binding protein is obtained. Ion exchange separation includes any method by which two substances are separated based on the difference in their respective ionic charges, and can employ either cationic exchange material or anionic exchange material.

The use of a cationic exchange material versus an anionic exchange material is based on the overall charge of the protein. Therefore, it is within the scope of this invention to employ an anionic exchange step prior to the use of a cationic exchange step, or a cationic exchange step prior to the use of an anionic exchange step. Furthermore, it is within the scope of this invention to employ only a cationic exchange step, only an anionic exchange step, or any serial combination of the two.

In performing the separation, the initial binding protein mixture can be contacted with the ion exchange material by using any of a variety of techniques, e.g., using a batch purification technique or a chromatographic technique.

For example, in the context of batch purification, ion exchange material is prepared in, or equilibrated to, the desired starting buffer. Upon preparation, or equilibration, a slurry of the ion exchange material is obtained. The binding protein solution is contacted with the slurry to adsorb the binding protein to be separated to the ion exchange material. The solution comprising the HCP(s) that do not bind to the ion exchange material is separated from the slurry, e.g., by allowing the slurry to settle and removing the supernatant. The slurry can be subjected to one or more wash steps. If desired, the slurry can be contacted with a solution of higher conductivity to desorb HCPs that have bound to the ion exchange material. In order to elute bound polypeptides, the salt concentration of the buffer can be increased.

Ion exchange chromatography may also be used as an ion exchange separation technique. Ion exchange chromatography separates molecules based on differences between the overall charge of the molecules. For the purification of a binding protein, the binding protein must have a charge opposite to that of the functional group attached to the ion exchange material, e.g., resin, in order to bind. For example, binding proteins which generally have an overall positive charge in the buffer pH below its pI, will bind well to cation exchange material, which contain negatively charged functional groups.

In ion exchange chromatography, charged patches on the surface of the solute are attracted by opposite charges attached to a chromatography matrix, provided the ionic strength of the surrounding buffer is low. Elution is generally achieved by increasing the ionic strength (i.e., conductivity) of the buffer to compete with the solute for the charged sites of the ion exchange matrix. Changing the pH and thereby altering the charge of the solute is another way to achieve elution of the solute. The change in conductivity or pH may be gradual (gradient elution) or stepwise (step elution).

Anionic or cationic substituents may be attached to matrices in order to form anionic or cationic supports for chromatography. Non-limiting examples of anionic exchange substituents include diethylaminoethyl (DEAE), quaternary aminoethyl(QAE) and quaternary amine(Q) groups. Cationic substitutents include carboxymethyl (CM), sulfoethyl(SE), sulfopropyl(SP), phosphate(P) and sulfonate (S). Cellulose ion exchange resins such as DE23™, DE32™, DE52™, CM-23™, CM-32™, and CM-52™ are available from Whatman Ltd. Maidstone, Kent, U.K. SEPHADEX®-based and -locross-linked ion exchangers are also known. For example, DEAE-, QAE-, CM and SP-SEPHADEX® and DEAE Q CM- and S-SEPHAROSE® and SEPHAROSE® Fast Flow are all available from Pharmacia AB. Further, both DEAE and CM derivitized ethylene glycol-methacrylate copolymer such as TOYOPEARL™ DEAE-6505 or M and TOYOPEARL™ CM-650S or M are available from Toso Haas Co., Philadelphia, Pa.

A mixture comprising a binding protein and impurities, e.g., HCP(s), is loaded onto an ion exchange column, such as a cation exchange column. For example, but not by way of limitation, the mixture can be loaded at a load of about 80 g protein/L resin depending upon the column used. An example of a suitable cation exchange column is a 80 cm diameter×23 cm long column whose bed volume is about 116 L. The mixture loaded onto this cation column can subsequently be washed with wash buffer (equilibration buffer). The binding protein is then eluted from the column, and a first eluate is obtained.

This ion exchange step facilitates the capture of the binding protein of interest while reducing impurities such as HCPs. In certain aspects, the ion exchange column is a cation exchange column. For example, but not by way of limitation, a suitable resin for such a cation exchange column is CM HyperDF resin. These resins are available from commercial sources such as Pall Corporation. This cation exchange procedure can be carried out at or around room temperature. This ion exchange step may also be combined with a hydrophobic interaction chromatographic process performed with resins having an ion exchange function and a hydrophobic interaction function.

In certain embodiments, the AEX or the AEX-MM flow-through eluate is further polished through a CEX-MM column. Non-limiting examples of CEX-MM resins include Capto MMC™, Capto MMC™ ImpRes (GE Healthcare, UK), Nuvia™ cPrime™ (Biorad, CA), Toyopearl MX Trp-650M (Tosoh Bioscience). In certain embodiments, Capto MMC™ and Capto MMC™ ImpRes resins are used. In other embodiments, Nuvia™ cPrime resin is used. In one embodiment, the CEX-MM column is equilibrated with Tris buffer at pH 7 and conductivity about 3-20 mS/cm followed by loading of DVD-Ig™ feed that was pre-adjusted to similar pH and conductivity of the equilibration buffer. In another embodiment, the CEX-MM column is equilibrated with sodium acetate buffer at pH 5 and conductivity about 3-20 mS/cm followed by loading DVD-Ig™ feed that was pre-adjusted to similar pH and conductivity of the equilibration buffer. The preferred equilibration buffer and feed conductivity is about 10-20 mS/cm, while the preferred equilibration buffer and feed pH is about 1-2.5 pH units lower than the pI of the binding protein or DVD-Ig. In one embodiment, the protein feed was supplemented with about 50 mM arginine. In one embodiment, the CEX-MM column was challenged with protein feed at a loading level up to about 400 g/L. In another embodiment, the CEX-MM column was challenged with protein feed at a loading level up to about 1200 g/L. In certain aspects, the column was run at a flow rate corresponding to about 3 min residence times (RT).

In certain aspects of the invention, the eluate from the AEX and/or AEX-MM chromatography and the CEX-MM chromatography step is subjected to filtration for the removal of viral particles, including intact viruses, if present. A non-limiting example of a suitable filter is the ViroSart CPV filter from Sartorius Other viral filters can be used in this filtration step and are well known to those skilled in the art. The eluate is passed through a pre-wetted filter of about 0.1 μm and a ViroSart CPV filter train at around 32 psig. Another non-limiting example of a suitable filter is the Ultipor DV50™ filter from Pall Corporation. Other viral filters can be used in this filtration step and are well known to those skilled in the art. The eluate is passed through a pre-wetted filter of about 0.1 μm and a 2×30-inch Ultipor DV50™ filter train at around 34 psig. In certain embodiments, following the filtration process, the filter is washed using, e.g., the elution buffer in order to remove any DVD-Ig™ retained in the filter housing. The filtrate can be stored in a pre-sterilized container at around 2-12° C.

In certain embodiments, the protein A eluate can be further purified using a cation exchange column. In certain embodiments, the equilibrating buffer used in the cation exchange column is a buffer having a pH of about 5.0. An example of a suitable buffer is about 210 mM sodium acetate, pH 5.0. Following equilibration, the column is loaded with sample prepared from the primary recovery step above. The column is packed with a cation exchange resin, such as CM Sepharose™ Fast Flow from GE Healthcare. The column is then washed using the equilibrating buffer. The column is next subjected to an elution step using a buffer having a greater ionic strength as compared to the equilibrating or wash buffer. For example, a suitable elution buffer can be about 790 mM sodium acetate, pH 5.0. The binding proteins will be eluted and can be monitored using a UV spectrophotometer set at OD₂₈₀ In a particular example, elution collection can be from upside 3 OD₂₈₀ to downside 8 OD₂₈₀ It should be understood that one skilled in the art may vary the conditions and yet still be within the scope of the invention.

In certain embodiments the Protein A eluate is instead further purified using an anion exchange column or membrane. A non-limiting example of a suitable column for this step is a 60 cm diameter×30 cm long column whose bed volume is about 85 L. The column is packed with an anion exchange resin, such as Q Sepharose™ Fast Flow from GE Healthcare. The column can be equilibrated using about seven column volumes of an appropriate buffer such as Tris/sodium chloride. An example of suitable conditions is 25 mM Tris, 50 mM sodium chloride at pH 8.0. Non-limiting examples of membrane products that feature anionic functions that are available commercially include QyuSpeed™ D (QSD) membrane absorber (Ashi Kasei, Japan) and Sartobind Q membrane absorber (Sartorious AG, Germany). The feed, pH and conductivity are adjusted to target values of pH from about 5-9 and conductivities of between 3-15 mS/cm. The membrane is equilibrated with a wash buffer such as 20 mM Tris, pH 8.5. The membrane is challenged with the feed at a loading level of 1-3 kg/L. After loading the column is flushed with 1-5 column volumes of equilibration buffer and the flow through and wash fractions collected and measured for protein concentrations by UV₂₈₀.

As noted above, accurate tailoring of a purification scheme relies on consideration of the protein to be purified. In certain embodiments, the separation steps of the instant invention are employed to separate a binding protein from one or more HCPs. Particular embodiments of the present invention feature DVD-Igs™ purification using cation exchanger-based mixed mode (CEX-MM) flow-through chromatography. The DVD-Ig™ is separated from impurities when the impurities specifically adhere to the CEX-MM resin and the DVD-Ig™ does not, i.e., the DVD-Ig™ is present in the flow through. This CEX-MM flow-through polishing step provides substantially higher throughput than conventional bind-elute method, hence greatly improving process efficiency and economics. The CEX-MM chromatography step can be used in post-protein A capture step in combination with other chromatographic steps to achieve target product quality. In one embodiment, the CEX-MM flow-through polishing step is used after Protein A capture and AEX flow-through chromatography step. In another embodiment, the CEX-MM flow-through polishing is used after Protein A capture and AEX-MM flow-through chromatography steps. In another embodiment, the CEX-MM flow-through polishing is used after Protein A capture, AEX flow-through and AEX-MM flow-through chromatography steps. In another embodiment, an AEX polishing step follows Protein A capture and CEX-MM flow-through polishing. In one embodiment, an AEX-MM polishing step follows Protein A capture, CEX-MM flow-through polishing and AEX flow-through polishing steps. Yet in another embodiment, an AEX-MM polishing step follows Protein A capture and CEX-MM flow-through polishing steps.

In certain embodiments, the anion exchange eluate is next filtered using, e.g., a 30-inch 0.45/0.2 μm Sartopore™ bi-layer filter cartridge. The ion exchange elution buffer can be used to flush the residual volume remaining in the filters and prepared for viral filtration and/or ultrafiltration/diafiltration.

In order to accomplish the ultrafiltration/diafiltration step, the filtration media is prepared in a suitable buffer, e.g., 20 mM sodium phosphate, pH 7.0. A salt such as sodium chloride can be added to increase the ionic strength, e.g., 100 mM sodium chloride. This ultrafiltration/diafiltration step serves to concentrate the binding proteins, remove the sodium acetate, and adjust the pH. Commercial filters are available to effectuate this step. For example, Millipore manufactures a 30 kD molecular weight cut-off (MWCO) cellulose ultrafilter membrane cassette. This filtration procedure can be conducted at or around room temperature.

In certain embodiments, the sample from the capture filtration step above is subjected to a second ion exchange separation step. Preferably this second ion exchange separation will involve separation based on the opposite charge of the first ion exchange separation. For example, if an anion exchange step is employed after primary recovery, the second ion exchange chromatographic step may be a cation exchange step. Conversely, if the primary recovery step was followed by a cation exchange step, that step would be followed by an anion exchange step. In certain embodiments the first ion exchange eluate can be subjected directly to the second ion exchange chromatographic step where the first ion exchange eluate is adjusted to the appropriate buffer conditions. Suitable anionic and cationic separation materials and conditions are described above.

The present invention may also features methods for producing a HCP-reduced binding protein preparation from a mixture comprising a binding protein and at least one HCP further comprising a hydrophobic interaction separation step. For example, a first eluate obtained from an ion exchange column can be subjected to a hydrophobic interaction material such that a second eluate having a reduced level of HCP is obtained. Hydrophobic interaction chromatography steps, such as those disclosed herein, are generally performed to remove protein aggregates, such as binding protein aggregates, and process-related impurities. Hydrophobic interaction chromatography steps can be performed simultaneously with ion exchange chromatography steps with chromatography resin having both ion exchange functions and hydrophobic functions. Such resins are characterized as mixed mode chromatography resins.

In performing the separation, the sample mixture is contacted with the HIC material, e.g., using a batch purification technique or using a column. Prior to HIC purification it may be desirable to remove any chaotropic agents or very hydrophobic substances, e.g., by passing the mixture through a pre-column.

For example, in the context of batch purification, HIC material is prepared in or equilibrated to the desired equilibration buffer. A slurry of the HIC material is obtained. The binding protein solution is contacted with the slurry to adsorb the antibody to be separated to the HIC material. The solution comprising the HCPs that do not bind to the HIC material is separated from the slurry, e.g., by allowing the slurry to settle and removing the supernatant. The slurry can be subjected to one or more washing steps. If desired, the slurry can be contacted with a solution of lower conductivity to desorb binding proteins that have bound to the HIC material. In order to elute bound binding proteins, the salt concentration can be decreased.

Whereas ion exchange chromatography relies on the charges of the binding proteins to isolate them, hydrophobic interaction chromatography uses the hydrophobic properties of the binding proteins. Hydrophobic groups on the binding protein interact with hydrophobic groups on the column. The more hydrophobic a protein is the stronger it will interact with the column. Thus the HIC step removes host cell derived impurities (e.g., DNA and other high and low molecular weight product-related species).

Hydrophobic interactions are strongest at high ionic strength, therefore, this form of separation is conveniently performed following salt precipitations or ion exchange procedures. Adsorption of the binding protein to a HIC column is favored by high salt concentrations, but the actual concentrations can vary over a wide range depending on the nature of the binding protein and the particular HIC ligand chosen. Various ions can be arranged in a so-called soluphobic series depending on whether they promote hydrophobic interactions (salting-out effects) or disrupt the structure of water (chaotropic effect) and lead to the weakening of the hydrophobic interaction. Cations are ranked in terms of increasing salting out effect as Ba++; Ca++; Mg++; Li+; Cs+; Na+; K+; Rb+; NH4+, while anions may be ranked in terms of increasing chaotropic effect as PO43−; SO42−; CH3CO3−; Cl−; Br−; NO3−; C1O4−; I−; SCN−.

In general, Na, K or NH4 sulfates effectively promote ligand-protein interaction in HIC. Salts may be formulated that influence the strength of the interaction as given by the following relationship: (NH4)2SO4>Na2SO4>NaCl>NH4C1>NaBr>NaSCN. In general, salt concentrations of between about 0.75 and about 2 M ammonium sulfate or between about 1 and 4 M NaCl are useful.

HIC columns normally comprise a base matrix (e.g., cross-linked agarose or synthetic copolymer material) to which hydrophobic ligands (e.g., alkyl or aryl groups) are coupled. A suitable HIC column comprises an agarose resin substituted with phenyl groups (e.g., a Phenyl Sepharose™ column) Many HIC columns are available commercially. Examples include, but are not limited to, TSKgel butyl NPR (Tosoh Bioscience LLC, King of Prussia, Pa.); Phenyl Sepharose™ 6 Fast Flow column with low or high substitution (Pharmacia LKB Biotechnology, AB, Sweden); Phenyl Sepharose™ High Performance column (Pharmacia LKB Biotechnology, AB, Sweden); Octyl Sepharose™ High Performance column (Pharmacia LKB Biotechnology, AB, Sweden); Fractogel™ EMD Propyl or Fractogel™ EMD Phenyl columns (E. Merck, Germany); Macro-Prep™ Methyl or Macro-Prep™ t-Butyl Supports (Bio-Rad, California); WP HI-Propyl (C3) column (J T Baker, New Jersey); and Toyopearl™ ether, phenyl or butyl columns (TosoHaas, PA).

In certain embodiments of the instant invention the sample containing DVD-Ig™ will be further processed using a hydrophobic interaction separation step. A non-limiting example of a suitable column for such a step is an 80 cm diameter×15 cm long column whose bed volume is about 75 L, which is packed with an appropriate resin used for HIC such as, but not limited to, Phenyl HP Sepharose™ from Amersham Biosciences, Upsala, Sweden. The flow-through preparation obtained from the previous anion exchange chromatography step comprising the DVD-Ig™ of interest can be diluted with an equal volume of around 1.7 M ammonium sulfate, 50 mM sodium phosphate, pH 7.0. This then can be subjected to filtration using a 0.45/0.2 μm Sartopore™ 2 bi-layer filter, or its equivalent. In certain embodiments, the hydrophobic chromatography procedure involves two or more cycles.

In certain embodiments, the HIC column is first equilibrated using a suitable buffer. A non-limiting example of a suitable buffer is 0.85 M ammonium sulfate, 50 mM sodium phosphate, pH 7.0. One skilled in the art can vary the equilibrating buffer and still be within the scope of the present invention by altering the concentrations of the buffering agents and/or by substituting equivalent buffers. In certain embodiments the column is then loaded with an anion exchange flow-through sample and washed multiple times, e.g., three times, with an appropriate buffer system such as ammonium sulfate/sodium phosphate. An example of a suitable buffer system includes 1.1 M ammonium sulfate, 50 mM sodium phosphate buffer with a pH of around 7.0. Optionally, the column can undergo further wash cycles. For example, a second wash cycle can include multiple column washes, e.g., one to seven times, using an appropriate buffer system. A non-limiting example of a suitable buffer system includes 0.85 M ammonium sulfate, 50 mM sodium phosphate, pH 7.0. In one aspect, the loaded column undergoes yet a third wash using an appropriate buffer system. The column can be washed multiple times, e.g., one to three times, using a buffer system such as 1.1 M ammonium sulfate, 50 mM sodium phosphate at a pH around 7.0. Again, one skilled in the art can vary the buffering conditions and still be within the scope of the present invention.

The column is eluted using an appropriate elution buffer. A suitable example of such an elution buffer is 0.5 M ammonium sulfate, 15 mM sodium phosphate at a pH around 7.0. The DVD-Ig™ of interest can be detected and collected using a conventional spectrophotometer from the upside at 3 OD₂₈₀ to downside of peak at 3 OD₂₈₀. Certain embodiments feature a hydrophobic interaction media with cationic exchange feature which is operated in flow through mode. The feed is pH and conductivity adjusted to target values of pH 5-7, 3-15 mS/cm and diluted to about 10-12 g/L. The resin is packed in a 1 ml column and equilibrated with 50 mM Na acetate, ph5, 5.5 or 6 buffer or 20 mM Tris, pH 7 buffer, each of the buffers with NaCl to match the load conductivity. The column is challenged with the feed at a resin loading level of 200-400 g/L and at a 0.3 ml/min flow rate. After loading the column is flushed with 15 column volumes of equilibration buffer and the flow through and wash fractions collected and measured for protein concentrations by UV280 and SEC methods in essentially a flow-through process.

In certain aspects of the invention, the eluate from the hydrophobic chromatography step is subjected to filtration for the removal of viral particles, including intact viruses, if present. A non-limiting example of a suitable filter is the Ultipor DV50□ filter from Pall Corporation. Other viral filters can be used in this filtration step and are well known to those skilled in the art. The HIC eluate is passed through a pre-wetted filter of about 0.1 nm and a 2×30-inch Ultipor DV50□ filter train at around 34 psig. In certain embodiments, following the filtration process, the filter is washed using, e.g., the HIC elution buffer in order to remove any binding proteins retained in the filter housing. The filtrate can be stored in a pre-sterilized container at around 12° C.

In certain embodiments viral reduction/inactivation can be achieved via the use of suitable filters. In certain embodiments viral filters with a nominal pore size of 20 nm are used. In certain embodiments the viral filters comprise a polyestersulfone (PES), polyvinylidene fluoride (PVDF), or a cellulose material. In certain embodiments, the viral filter is flushed/equilibrated with a suitable buffer at a suitable pH prior to sample loading. A non-limiting sample of a suitable buffer is 50 mM NaAc or 25 mM trolamine. A non-limiting example of a suitable pH is about 4, about 5, about 6, about 7, about 8, or about 9, or any pH within this range of measurements. In certain embodiments the filter achieves at least 30% throughput recovery of the target binding protein. In other embodiments the filter achieves at least 40% throughput recovery, at least 50% throughput recovery, at least 60% throughput recover, at least 70% throughput recovery, at least 80% throughput recovery, at least 90% throughput recovery, or 100% throughput recovery. In certain embodiments the filter achieves a flow (flux) of 0-600 liters/hour/meter2 (LMH). In certain embodiments the filter percent flux decay is 0-100%. A non-limiting example of a suitable filter is the Ultipor□ DV20 filter from Pall Corporation. In certain embodiments, alternative filters are employed for viral reduction, such as, but not limited to, Viresolve™ filters (Millipore, Billerica, Mass.); Virosart™ filters (Sartorius, Bohemia, N.Y.); Zeta Plus VR™ filters (CUNO, Meriden, Conn.); and Planova™ filters (Asahi Kasei Pharma, Planova Division, Buffalo Grove, Ill.).

In a certain embodiment, the filtrate from the above is again subjected to ultrafiltration/diafiltration. Ultrafiltration is described in detail in: Microfiltration and Ultrafiltration: Principles and Applications, L. Zeman and A. Zydney (Marcel Dekker, Inc., New York, N.Y., 1996); and in: Ultrafiltration Handbook, Munir Cheryan (Technomic Publishing, 1986; ISBN No. 87762-456-9). A preferred filtration process is Tangential Flow Filtration as described in the Millipore catalogue entitled “Pharmaceutical Process Filtration Catalogue” pp. 177-202 (Bedford, Mass., 1995/96). Ultrafiltration is generally considered to mean filtration using filters with a pore size of smaller than 0.1 nm. By employing filters having such small pore size, the volume of the sample can be reduced through permeation of the sample buffer through the filter while antibodies are retained behind the filter.

Diafiltration is a method of using ultrafilters to remove and exchange salts, sugars, and non-aqueous solvents, to separate free from bound species, to remove low molecular-weight material, and/or to cause the rapid change of ionic and/or pH environments. Microsolutes are removed most efficiently by adding solvent to the solution being ultra-filtered at a rate approximately equal to the ultrafiltration rate. This washes microspecies from the solution at a constant volume, effectively purifying the retained binding protein. In certain embodiments of the present invention, a diafiltration step is employed to exchange the various buffers used in connection with the instant invention, optionally prior to further chromatography or other purification steps, as well as to remove impurities from the binding protein preparations. This step is important if a practitioner's end point is to use the DVD-Ig™ in a pharmaceutical formulation. This process pre-concentrate the DVD-Ig™ to an intermediate target concentration and formulate it in the desired formulation buffer. In certain embodiments, continuous diafiltration with multiple volumes, e.g., two to eight volumes, of a formulation buffer is performed. A non-limiting example of a suitable formulation buffer is 15 mM histidine, pH 6.0 buffer. Another non-limiting example of a suitable formulation buffer is 5 mM methionine, 2% mannitol, 0.5% sucrose, pH 5.9 buffer (no Tween). Upon completion of this diavolume exchange the DVD-Ig™ are further concentrated. Once a predetermined concentration of DVD-Ig™ has been achieved, the system is rinsed with specific amount of diafiltration buffer to recover the retaining proteins in the system and to meet the formulation protein concentration target. In another embodiment, once a predetermined concentration of DVD-Ig™ has been achieved, a practitioner can calculate the amount of 10% Tween that should be added to arrive at a final Tween concentration of about 0.005% (v/v).

Certain embodiments of the present invention will include further purification steps. Examples of additional purification procedures which can be performed prior to, during, or following the ion exchange chromatography method include ethanol precipitation, isoelectric focusing, reverse phase HPLC, chromatography on silica, chromatography on heparin Sepharose™, further anion exchange chromatography and/or further cation exchange chromatography, chromatofocusing, SDS-PAGE, ammonium sulfate precipitation, hydroxylapatite chromatography, gel electrophoresis, dialysis, and affinity chromatography (e.g., using protein G, an antibody, a specific substrate, ligand or antigen as the capture reagent).

### 5. METHODS OF ASSAYING SAMPLE PURITY

### 5.1 Assaying Host Cell Protein

The present invention also provides methods for determining the residual levels of host cell protein (HCP) concentration in the isolated/purified DVD-Ig™ composition. As described above, HCPs are desirably excluded from the final target substance product. Exemplary HCPs include proteins originating from the source of the DVD-Ig™ production. Failure to identify and sufficiently remove HCPs from the target DVD-Ig™ may lead to reduced efficacy and/or adverse subject reactions.

As used herein, the term “HCP ELISA” refers to an ELISA where the second antibody used in the assay is specific to the HCPs produced from cells, e.g., CHO cells, used to generate the DVD-Ig. The second antibody may be produced according to conventional methods known to those of skill in the art. For example, the second antibody may be produced using HCPs obtained by sham production and purification runs, i.e., the same cell line used to produce the antibody of interest is used, but the cell line is not transfected with DVD-Ig™ DNA. In an exemplary embodiment, the second antibody is produced using HPCs similar to those expressed in the cell expression system of choice, i.e., the cell expression system used to produce the target DVD-Ig.

Generally, HCP ELISA comprises sandwiching a liquid sample comprising HCPs between two layers of antibodies, i.e., a first antibody and a second antibody. The sample is incubated during which time the HCPs in the sample are captured by the first antibody, for example, but not limited to goat anti-CHO, affinity purified (Cygnus). A labeled second antibody, or blend of antibodies, specific to the HCPs produced from the cells used to generate the antibody, e.g., anti-CHO HCP biotinylated, is added, and binds to the HCPs within the sample. In certain embodiments the first and second antibodies are polyclonal antibodies. In certain aspects the first and second antibodies are blends of polyclonal antibodies raised against HCPs, for example, but not limited to biotinylated goat anti Host Cell Protein Mixture 599/626/748. The amount of HCP contained in the sample is determined using the appropriate test based on the label of the second antibody.

HCP ELISA may be used for determining the level of HCPs in a binding protein composition, such as an eluate or flow-through obtained using the process described above. The present invention also provides a composition comprising a binding protein, wherein the composition has no detectable level of HCPs as determined by an HCP Enzyme Linked Immunosorbent Assay (“ELISA”).

### 5.2 Assaying Affinity Chromatographic Material

In certain embodiments, the present invention also provides methods for determining the residual levels of affinity chromatographic material in the isolated/purified binding protein composition. In certain contexts such material leaches into the binding protein composition during the purification process. In certain embodiments, an assay for identifying the concentration of Protein A in the isolated/purified binding protein composition is employed. As used herein, the term “Protein A ELISA” refers to an ELISA where the second antibody used in the assay is specific to the Protein A employed to purify the DVD-Ig™ of interest. The second antibody may be produced according to conventional methods known to those of skill in the art. For example, the second antibody may be produced using naturally occurring or recombinant Protein A in the context of conventional methods for antibody generation and production.

Generally, Protein A ELISA comprises sandwiching a liquid sample comprising Protein A (or possibly containing Protein A) between two layers of anti-Protein A antibodies, i.e., a first anti-Protein A antibody and a second anti-Protein A antibody. The sample is exposed to a first layer of anti-Protein A antibody, for example, but not limited to polyclonal antibodies or blends of polyclonal antibodies, and incubated for a time sufficient for Protein A in the sample to be captured by the first antibody. A labeled second antibody, for example, but not limited to polyclonal antibodies or blends of polyclonal antibodies, specific to the Protein A is then added, and binds to the captured Protein A within the sample. Additional non-limiting examples of anti-Protein A antibodies useful in the context of the instant invention include chicken anti-Protein A and biotinylated anti-Protein A antibodies. The amount of Protein A contained in the sample is determined using the appropriate test based on the label of the second antibody. Similar assays can be employed to identify the concentration of alternative affinity chromatographic materials.

Protein A ELISA may be used for determining the level of Protein A in a binding protein composition, such as an eluate or flow-through obtained using the process described in above. The present invention also provides a composition comprising a binding protein, wherein the composition has no detectable level of Protein A as determined by an Protein A Enzyme Linked Immunosorbent Assay (“ELISA”).

### 6. FURTHER MODIFICATIONS

The binding proteins of the present invention can be modified. In some embodiments, the binding proteins or antigen-binding fragments thereof are chemically modified to provide a desired effect. For example, pegylation of binding proteins or antigen binding fragments of the invention may be carried out by any of the pegylation reactions known in the art, as described, e.g., in the following references: Focus on Growth Factors 3:4-10 (1992); EP 0 154 316; and EP 0 401 384, each of which is incorporated by reference herein in its entirety. In one aspect, the pegylation is carried out via an acylation reaction or an alkylation reaction with a reactive polyethylene glycol molecule (or an analogous reactive water-soluble polymer). A suitable water-soluble polymer for pegylation of the binding proteins and antigen binding fragments of the invention is polyethylene glycol (PEG). As used herein, “polyethylene glycol” is meant to encompass any of the forms of PEG that have been used to derivatize other proteins, such as mono (Cl C10) alkoxy- or aryloxy-polyethylene glycol.

Methods for preparing pegylated binding proteins and antigen binding fragments of the invention will generally comprise the steps of (a) reacting the binding protein or antigen binding fragment with polyethylene glycol, such as a reactive ester or aldehyde derivative of PEG, under suitable conditions whereby the binding protein or antigen binding fragment becomes attached to one or more PEG groups, and (b) obtaining the reaction products. It will be apparent to one of ordinary skill in the art to select the optimal reaction conditions or the acylation reactions based on known parameters and the desired result.

Pegylated binding proteins and antigen binding fragments may generally be used to treat mammalian diseases and disorders. Generally the pegylated binding proteins and antigen binding fragments have increased half-life, as compared to the nonpegylated binding proteins and antigen binding fragments. The pegylated binding proteins and antigen binding fragments may be employed alone, together, or in combination with other pharmaceutical compositions.

A binding protein or antigen binding portion of the invention can be derivatized or linked to another functional molecule (e.g., another peptide or protein). Accordingly, the binding proteins and antigen binding portions of the invention are intended to include derivatized and otherwise modified forms described herein, including immunoadhesion molecules. For example, a binding protein or antigen binding portion of the invention can be functionally linked (by chemical coupling, genetic fusion, noncovalent association or otherwise) to one or more other molecular entities, such as another binding protein (e.g., a bispecific binding protein or a diabody), a detectable agent, a cytotoxic agent, a pharmaceutical agent, and/or a protein or peptide that can mediate associate of the binding protein or antigen binding portion with another molecule (such as a streptavidin core region or a polyhistidine tag).

One type of derivatized binding protein is produced by crosslinking two or more binding proteins (of the same type or of different types, e.g., to create bispecific binding proteins). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which a binding protein or antigen binding portion of the invention may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. A binding protein may also be derivatized with detectable enzymes, such as alkaline phosphatase, horseradish peroxidase, glucose oxidase and the like. When a binding protein is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. A binding protein may also be derivatized with biotin, and detected through indirect measurement of avidin or streptavidin binding.

### 7. PHARMACEUTICAL COMPOSITIONS

The DVD-Igs™ of the invention can be incorporated into pharmaceutical compositions suitable for administration to a subject. Typically, the pharmaceutical composition comprises a DVD-Ig™ of the invention and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it is desirable to include isotonic agents, e.g., sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the DVD-Ig™.

It should be understood that the DVD-Igs™ can be used alone or in combination with an additional agent, e.g., a therapeutic agent, with the additional agent being selected by the skilled artisan for its intended purpose. For example, the additional agent can be a therapeutic agent art-recognized as being useful to treat the disease or condition being treated by the DVD-Ig™ of the present invention. The additional agent also can be an agent which imparts a beneficial attribute to the therapeutic composition, e.g., an agent which affects the viscosity of the composition.

### EXAMPLES

### 1. Isolation and Purification of DVD-Igs Using Mixed-Mode Chromatography

### 1.1 Flow-Through Polishing of DVD1 by Capto MMC™ Resin

Example DVD1, a DVD-Ig™, which was generated from a process using MabSelect SuRe™ Protein A capture followed by Q membrane polishing, was used as the load material for Capto MMC™ flow-through processing. This feed contained 1.2-1.5% aggregates and was pH and conductivity adjusted to the targeted values (i.e. pH 5-7, 3-15 mS/cm) and then diluted to 10-12 g/L. The Capto MMC™ resin was packed in a 1 mL column, and was equilibrated with 50 mM Na acetate, pH 5, 5.5 or 6 buffer or 20 mM Tris, pH 7 buffer; each of these buffers contained proper amount of NaCl to match with the respective load conductivity. The column was challenged with each conditioned feed at a resin loading level of about 200-400 g/L and at a flow rate of 0.3 ml/min. After the loading, the column was flushed with 15 CV of equilibration buffer. The flow-through and wash fractions were collected and measured for protein concentrations by UV₂₈₀ and aggregate levels by SEC.

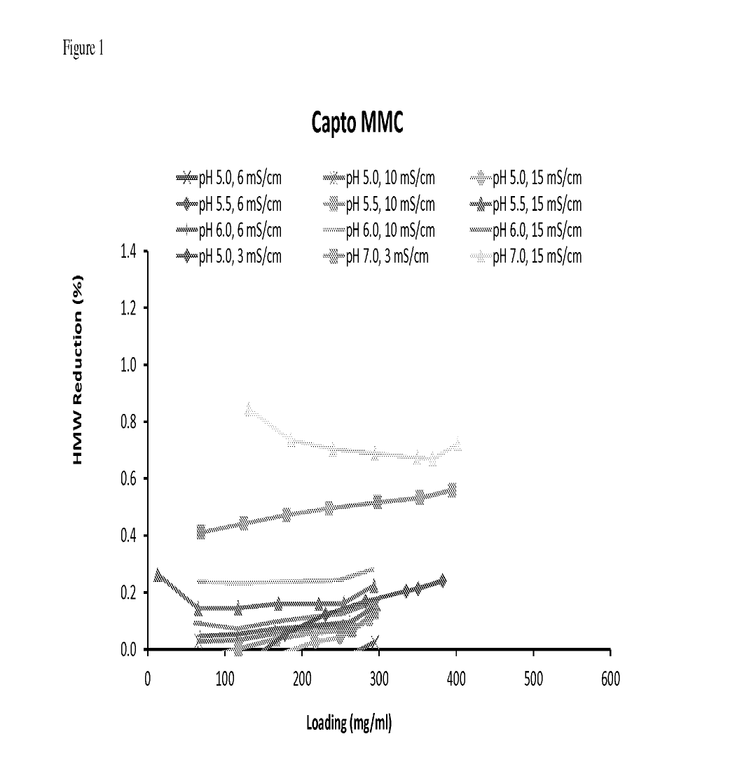

FIG. 1 summarizes the reduction of DVD1 aggregate levels upon flow-through polishing by Capto MMC™ resin under various pH and salt conditions. The data suggest that the best aggregate reduction occurs at the condition of neutral pH and high conductivity (i.e. pH 7, 15 mS/cm). This behavior is different from typical CEX resins which usually provide improved selectivity and better separation at lower pH and conductivity conditions.

### 1.2 Flow-Through Polishing of DVD1 by Capto MMC™ ImpRes Resin

A different lot of Example DVD1, again generated from a process using MabSelect SuRe™ Protein A capture followed by Q membrane polishing, was used as the feed for Capto MMC™ ImpRes flow-through evaluation. This feed contained about 1.8-2.2% aggregates, and was pH and conductivity adjusted to the target settings (i.e. pH 7, 3-20 mS/cm) and then diluted to about 10 g/L. The Capto MMC™ ImpRes resin was packed in a 1 mL column, and was equilibrated with 20 mM Tris, pH 7 buffer containing 10-200 mM NaCl (to match up with the respective load conductivity). The column was challenged with each conditioned feed at a resin loading level of about 400-600 g/L and at flow rate of 0.3 ml/min. After the loading, the column was flushed with 15 CV of equilibration buffer. The flow-through and wash fractions were collected and measured for protein concentrations by UV₂₈₀ and aggregate levels by SEC.

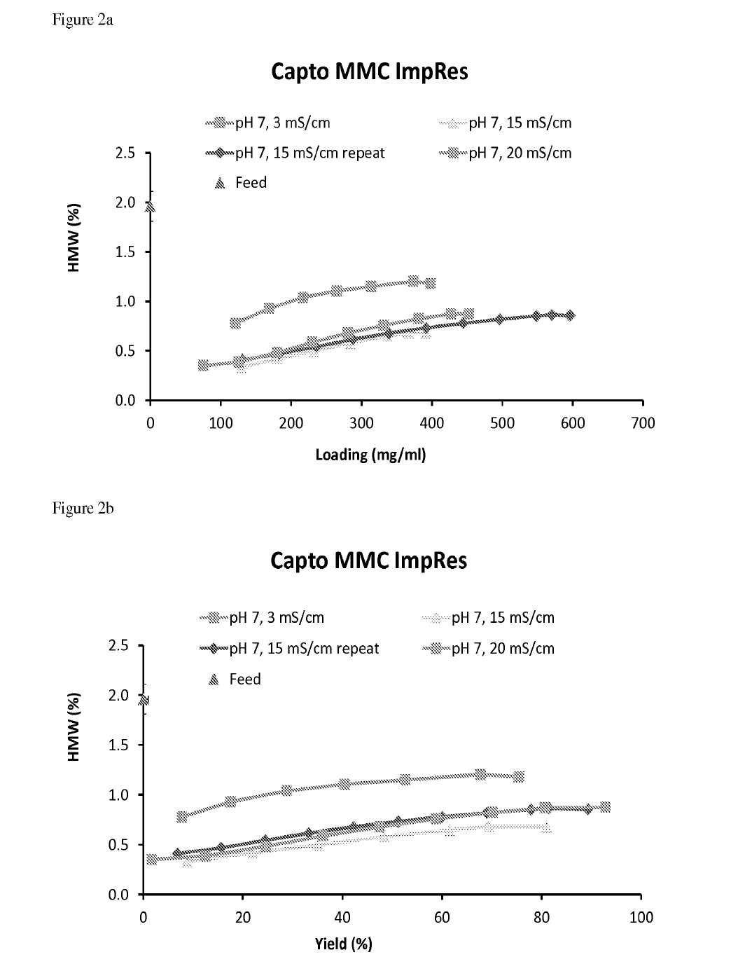

FIG. 2 shows cumulative aggregate levels for DVD1 as a function of resin loading (a) or cumulative yield (b) under different operating conditions. The data indicates that the aggregate levels in the flow-through pool increase gradually as the resin loading level increases. However, at a loading level of 600 g/L the aggregate level is still below 1% (e.g. at pH 7, 15 to 20 mS/cm). Altogether, a 90% product recovery was achieved, reflecting the high-throughput performance of this process.

Additional Capto MMC™ ImpRes flow-through polishing runs were performed using DVD1 Q flow-through pool (which was obtained from MabSelect SuRe™ Protein A eluate) with a 0.66 cm×10 cm column at pH 7, 15 mS/cm loading conditions. In this case, the feed was adjusted to a protein concentration of 6.3 g/L and had a starting aggregate level of about 0.7%. The Capto MMC™ ImpRes column was equilibrated with 20 mM Tris, 140 mM NaCl, pH 7 buffer followed by feeding at a loading level of up to 500 g/L at a flow rate of 1.2 ml/min. After loading, the column was washed with 15 CV of equilibration buffer prior to regeneration with 2M NaCl and cleaned with 0.5N NaOH. The flow-through and wash fractions were collected and analyzed by UV₂₈₀ and SEC.

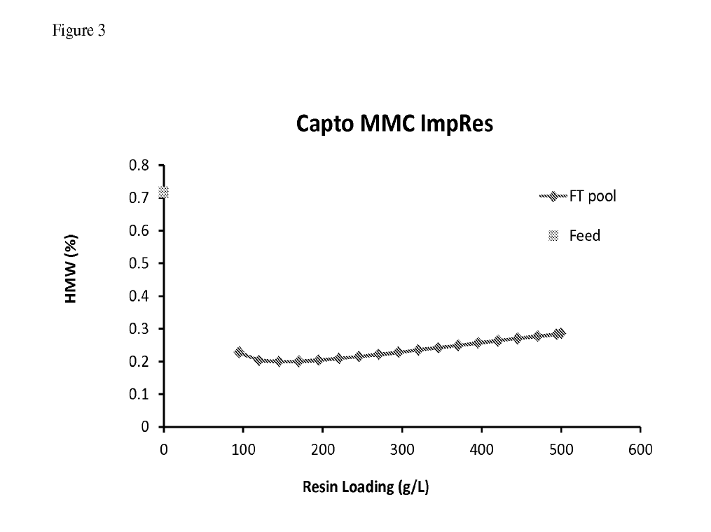

FIG. 3 shows DVD1 product pool aggregate levels as a function of resin loading at pH 7 and 15 mS/cm. Clearly, the aggregate levels in Capto MMC™ ImpRes flow-through pool can be reduced to as low as 0.3% with product recoveries ≧92%. A 20% reduction in DVD1 fragments was also observed. The overall process operation and performances are summarized in Table 2.

TABLE 2
DVD1 purification by Protein A → Q membrane FT →
Capto MMC ™ ImpRes FT process (Run 1)

| | Loading | Yield | HMW | Monomer | LMW |
|---|---|---|---|---|---|
| Step | Conditions | (%) | (%) | (%) | (%) |
| MabSelect | Clarified | 94 | 0.51 | 97.81 | 1.68 |
| SuRe ™ | harvest, 0.74 g/L | | | | |
| Protein A | | | | | |
| Capture | | | | | |
| CE50 Depth | pH 8.5, 5 | 97 | 2.87 | 95.69 | 1.44 |
| Filtration | mS/cm, 2 kg/m² | | | | |
| Sartobind Q | pH 8.5, 4.8 | 96 | 0.67 | 98.10 | 1.23 |
| membrane | mS/cm, 3 kg/L | | | | |
| Flow-through | | | | | |
| Capto MMC ™ | pH 7, 15 | 92 | 0.30 | 98.72 | 0.97 |
| ImpRes | mS/cm, 500 g/L | | | | |
| Flow-through | | | | | |

Table 3 shows the performance results for a second run (Run 2) of the same processing steps as shown in Table 1 but using a different lot of DVD1. In this experiment, the loading levels for the CE50 filter, Q membrane and Capto MMC™ ImpRes resins were somewhat different from the run described in Table 2. In this case, the Capto MMC™ ImpRes resin was able to reduce the aggregate level down to about 0.5% and HCP by about 3 fold.

TABLE 3
DVD1 purification by Protein A → Q membrane FT → Capto MMC ™ ImpRes
FT process (Run 2)

| | | Yield | HMW | Monomer | LMW | HCP |
|---|---|---|---|---|---|---|
| Step | Loading Conditions | (%) | (%) | (%) | (%) | (ng/mg) |
| MabSelect | Clarified harvest, | 81 | 1.51 | 97.21 | 1.28 | 2057 |
| SuRe ™ Protein A | 1.16 g/L | | | | | |
| Capture | | | | | | |
| CE50 Filtration/ | pH 8.5, 5.1 mS/cm, | 91 | 1.23 | 97.57 | 1.20 | 1657 |
| Sartobind Q | 1.7 kg/m² (CE50), | | | | | |
| membrane Flow- | 3.8 kg/L (Q memb.) | | | | | |
| through | | | | | | |
| Capto MMC ™ | pH 7, 15 mS/cm, | 88 | 0.54 | 98.19 | 1.27 | 595 |
| ImpRes Flow- | 578 g/L | | | | | |
| through | | | | | | |

Evaluation of Capto MMC™ ImpRes flow-through polishing run was also performed for DVD1 material which was processed using Protein A capture, Q membrane flow-through and Capto™ Adhere ImpRes flow-through. In this case the MabSelect SuRe™ Protein A eluate was supplemented with 100 mM arginine and then adjusted to pH 8.5 and 5 mS/cm at a protein concentration of 6.2 g/L. The conditioned Protein A eluate was then filtered through a Millistak CE50 depth filter at a loading level of about 100 L/m² or 613 g/m². The CE50 filtrate was further flowed through a Sartobind Q membrane adsorber at a membrane loading level of about 1 kg/L. The Q membrane flow-through pool was further flowed through a Capto™ Adhere ImpRes column at similar solution conditions and a resin loading level of about 236 g/L. The Capto™ Adhere ImpRes flow-through wash pool was conditioned to pH 7 and 15 mS/cm and used as the load for the Capto MMC™ ImpRes run. In this case, the Capto MMC™ ImpRes column was equilibrated with 20 mM Tris, 155 mM NaCl, pH 7 buffer and then loaded with feed to 475 g/L resin at a flow rate equal to 3.3 min RT. After loading, the column was washed with 15 CV of equilibration buffer prior to regeneration with 2M NaCl and cleaned with 0.5N NaOH. The flow-through and wash fractions were collected and analyzed by UV₂₈₀ and SEC.

Table 4 summarizes the four-step process performance for DVD1. The aggregate level was reduced from 4.24% down to 0.07%, and the HCP level reduced from about 7500 ng/mg to 4.4 ng/mg.

TABLE 4
DVD 1 purification by Protein A → Q membrane FT→ Capto ™ Adhere
ImpRes FT → Capto MMC ™ ImpRes FT process

| | | Yield | HMW | Monomer | LMW | HCP |
|---|---|---|---|---|---|---|
| Step | Loading Conditions | (%) | (%) | (%) | (%) | (ng/mg) |
| MabSelect | Clarified harvest, | 94 | 4.24 | 94.52 | 1.24 | 7504 |
| SuRe ™ Protein A | 0.74 g/L | | | | | |
| Capture | | | | | | |
| CE50 Depth | pH 8.5, 5 mS/cm, | 97 | 2.77 | 95.92 | 1.31 | 6408 |
| Filtration | 613 g/m² | | | | | |
| Sartobind Q | pH 8.5, 5 mS/cm, | 97 | 0.66 | 97.83 | 0.70 | 1229 |
| membrane Flow- | 1 kg/L | | | | | |
| through | | | | | | |
| Capto Adhere | pH 8.5, 5 mS/cm, | 88 | 0.15 | 99.20 | 0.65 | 7.6 |
| ImpRes Flow- | 236 g/L | | | | | |
| through | | | | | | |
| Capto MMC ™ | pH 7, 15 mS/cm, | 90 | 0.07 | 99.18 | 0.75 | 4.4 |
| ImpRes Flow- | 475 g/L | | | | | |
| through | | | | | | |

### 1.3 Flow-Through Polishing of DVD1 by Nuvia™ cPrime Resin

A different multimodal cation exchange resin, Nuvia cPrime, was also tested for flow-through polishing of DVD1. DVD1 BDS derived from Protein A capture and Q membrane polishing, containing 1.6% aggregates and adjusted to pH 7 and 15 mS/cm at 9.2 g/L, was used as the load. The Nuvia™ cPrime resin was packed in a 1 mL column, and was equilibrated with 20 mM Tris, 140 mM NaCl, pH 7 buffer and then loaded with feed to 400 g/L at a flow rate of 0.3 ml/min After the loading, the column was flushed with 15 CV of equilibration buffer. The flow-through and wash fractions were collected based on UV₂₈₀ reading 200-200 mAU, and were measured for protein concentrations and aggregate levels by SEC.

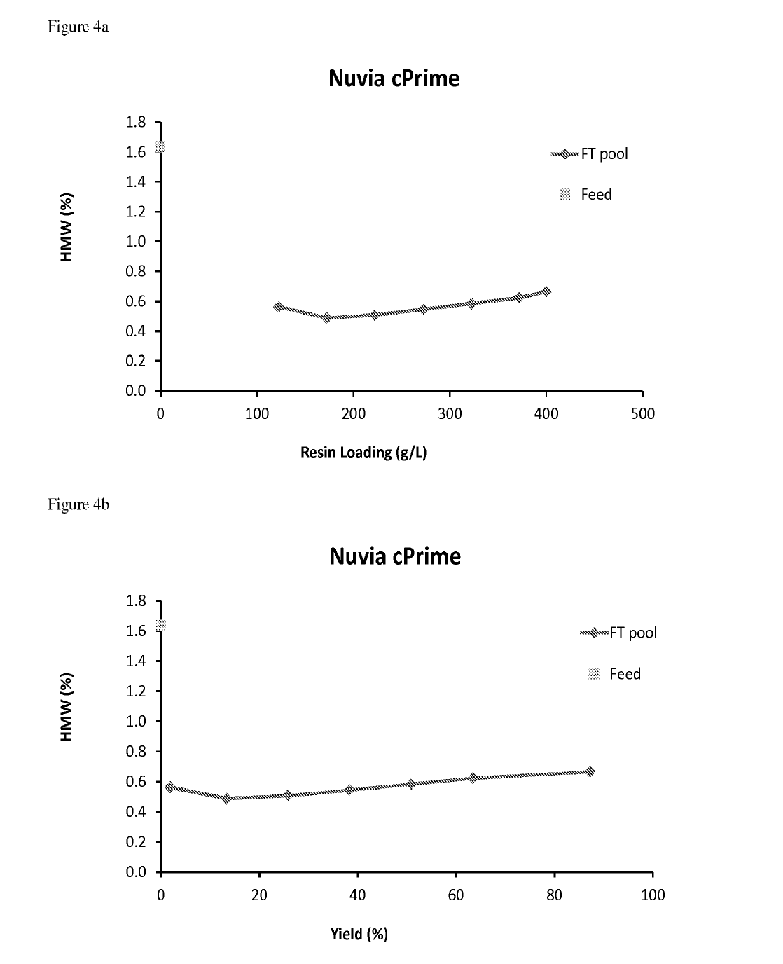

FIG. 4 shows cumulative aggregate levels for DVD1 as a function of resin loading (a) or cumulative yield (b) at pH 7 and 15 mS/cm. Like Capto MMC™ ImpRes resin, the Nuvia™ cPrime resin also showed significant aggregate clearance at high resin loading condition. The final product pool aggregates level was reduced to 0.68% (or 58% reduction) with 88% step yield, which may be further improved upon process optimization.

### 1.4 Flow-Through Polishing of DVD2 by Capto MMC™ ImpRes Resin

Example DVD 2, another DVD-Ig, was also purified by Capto MMC™ ImpRes flow-through polishing. Specifically, the load material for this study was generated from a process using ProSep Ultra Plus Protein A capture followed by Sartobind Q membrane polishing. Prior to loading the feed was pH and conductivity conditioned to pH 5-8.5, 3-16.5 mS/cm, and in some cases was also supplemented with 45 mM arginine. The starting load material had aggregate levels of 1.5-1.7% at a protein concentration of ˜10 g/L. A 1 mL HiTrap Capto MMC™ ImpRes column was used in these experiments. After equilibration the column was loaded with the respective feed at up to 1200 g/L at a flow equivalent to 3 min RT and then followed by a 20 CV equilibration buffer wash. The flow-through and wash fractions were collected and analyzed for protein concentrations and aggregate levels.

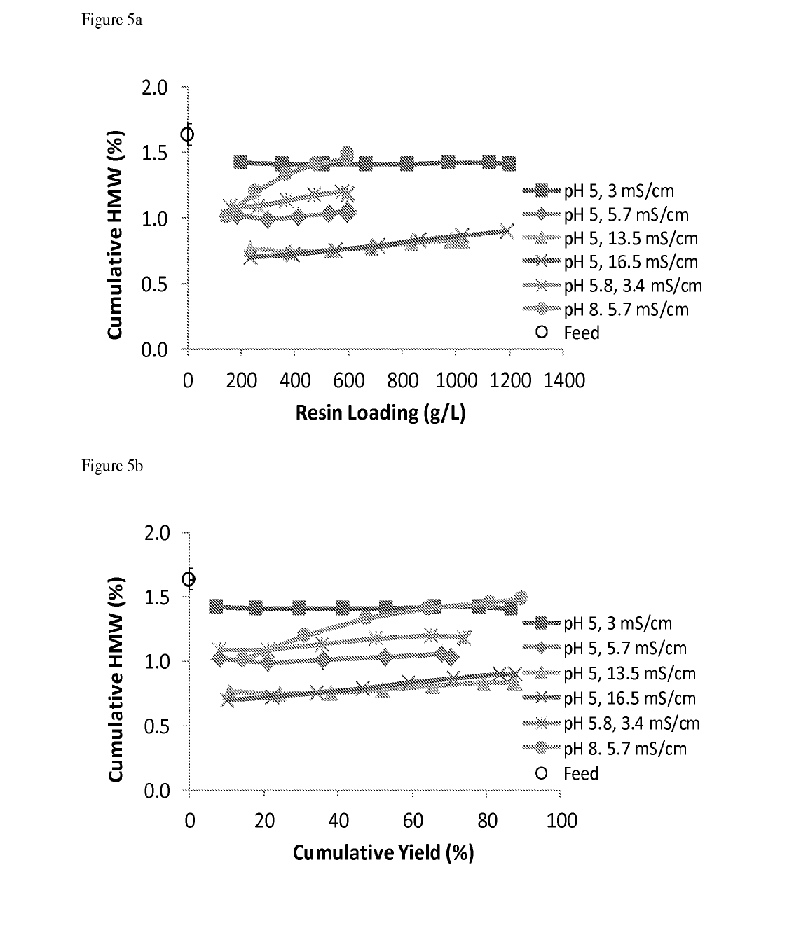

FIG. 5 shows DVD2 flow-through pool aggregate levels as a function of resin loading (a) or yield (b) under different operating conditions. Under the tested conditions, improved aggregate clearance was obtained when the feed was processed at higher conductivity and lower pH (e.g. 13.5-16.5 mS/cm at pH 5). Without further optimization, the DVD2 aggregate levels were significantly reduced even at resin loading levels over 1000 g/L where product recovery exceeded 90%.

### 1.5 Flow-Through Polishing of DVD2 by Nuvia™ cPrime Resin

The Nuvia™ cPrime resin was also tested for flow-through polishing of DVD2. DVD2 in-process feed stream derived from ProSep Ultra Plus Protein A capture and Q membrane polishing, containing 1.1% aggregates and 10 ng/mg HCP, and adjusted to pH 5 and 15 mS/cm at ˜8 g/L, was used as the load. The Nuvia™ cPrime resin was packed in a 1 ml, column, and was equilibrated with 25 mM Na acetate, 100 mM NaCl, 50 mM arginine, pH 5 buffer and then loaded with feed to 800-1000 g/L at a flow rate of 0.3 ml/min. After the loading, the column was flushed with 15 CV of equilibration buffer. The flow-through and wash fractions were collected based on UV₂₈₀ reading 200-200 mAU, and were measured for protein concentrations and aggregate levels by SEC.

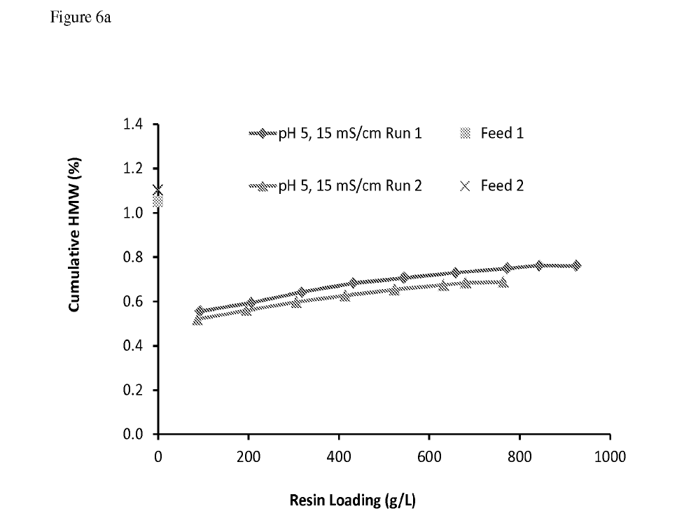

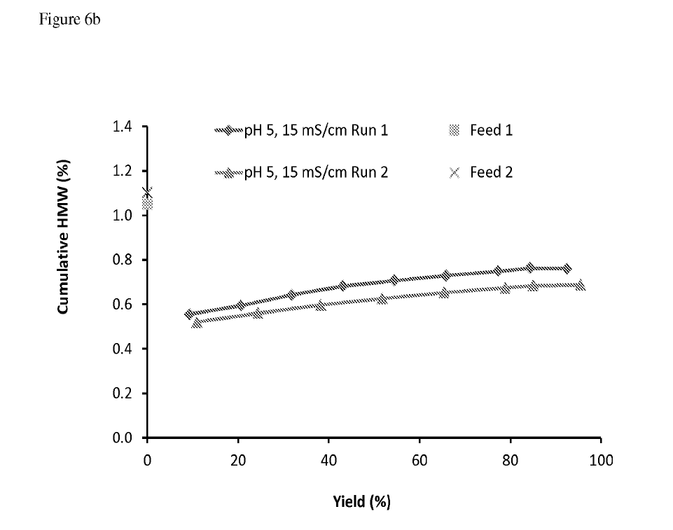

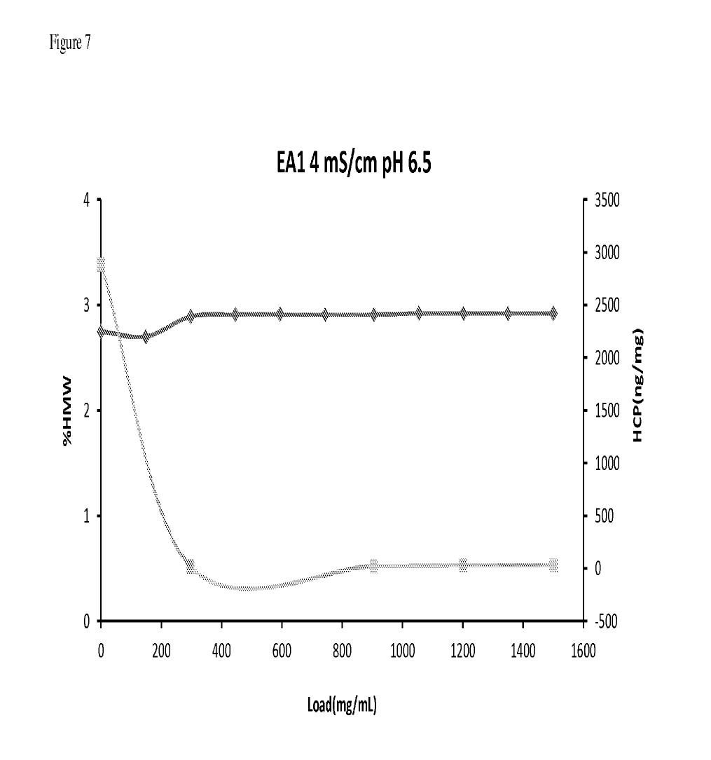

FIG. 6 shows cumulative aggregate levels for DVD2 as a function of resin loading (a) or cumulative yield (b) at pH 5 and 15 mS/cm. Like Capto MMC™ ImpRes resin, the Nuvia™ cPrime resin also showed significant aggregate clearance at high resin loading condition. The final product pool aggregates level was reduced by 40% with 95% step yield. Along with aggregate reduction, the product pool HCP level was also reduced to 1 ng/mg (or 10 fold reduction from the feed). Through process optimization the purification performance for this process can be further improved.

In summary, the salt-tolerant cation-exchange based mixed mode resins were explored for flow-through polishing of various DVD-Igs™. One exemplary process based on Protein A capture, Q membrane and Capto MMC™ ImpRes flow-through polishing demonstrated excellent product quality and high yield for different DVDs. This type of resin can remove product- and process-related impurities including aggregates (dimers), fragments, and HCPs when operating in negative chromatography (i.e. flow-through) mode. The method features substantially higher throughput (>10 fold) as compared to standard bind-elute operations, and as a manufacturing utility allows significant reduction in required column size, buffer consumption, and ultimately operating cost. These resins can be used in combination with other conventional chromatography methods to achieve desired protein product quality.

### 2. Isolation and Purification of DVD-Igs Using Anion Exchangers

### 2.1 DVD-Ig™ Polishing by QyuSpeed™ D (QSD) Membrane Absorber

Four DVD Igs, EA1, EA5, EA6 and EA7, were evaluated at bench scale for the ability of the QSD anion exchanger to remove aggregates and HCP. Protein A eluate of each DVD was used as the feed and the specific conditions are shown in Table 5. QyuSpeed™ D membrane adsorbers (cat# QDMY007, Lot 12Y06D006) were used for all runs. For each molecule, the filter was flushed with 30 ml of equilibration buffer (70 mM trolamine acetate pH 6.5 for pH 5.5 or 6.5 evaluations or 25 mM trolamine, 40 mM NaCl pH 8 for pH 8 evaluations) prior to use; 10 ml was flushed through device, and 20 ml was flushed across the membrane. pH and conductivity conditioned load (4 or 6 mS/cm; pH 5.5, 6.5, or 8.0) was then filtered through the membrane and fractionated by volume. Following filtration, the device was cleaned in reverse flow with 1M NaCl (regeneration), 20% ethanol, and sanitized with 1M NaOH.

TABLE 5
DVD-Ig ™ feed used for QSD flow-through runs

| | | Conductivity |
|---|---|---|
| Molecule | pH | mS/cm |
| EA1 | 6.5 | 4, 6, 8 |
| EA5 | 6.5 | 4.0 |
| EA6 | 6.5 | 4.0 |
| EA7 | 5.5, 6.5 | 4, 6 |

EA1

Protein A eluate (Batch 17098BI) was conditioned to 4 mS/cm and pH 6.5, then loaded over the QSD membrane. The results are shown in FIG. 7, where lines marked with diamonds represent aggregate reduction and lines marked with squares denote host cell protein (HCP) reduction. No aggregate reduction was noted. However, HCP was reduced from 2400 ng/mg to 30 ng/mg. The yield for this run was 97%. Thus, the QSD shows good HCP clearance and good recovery for EA1. Aggregate clearance for EA1 was also tested at pH 8 and 6 mS/cm, but no reduction was observed.

EA5

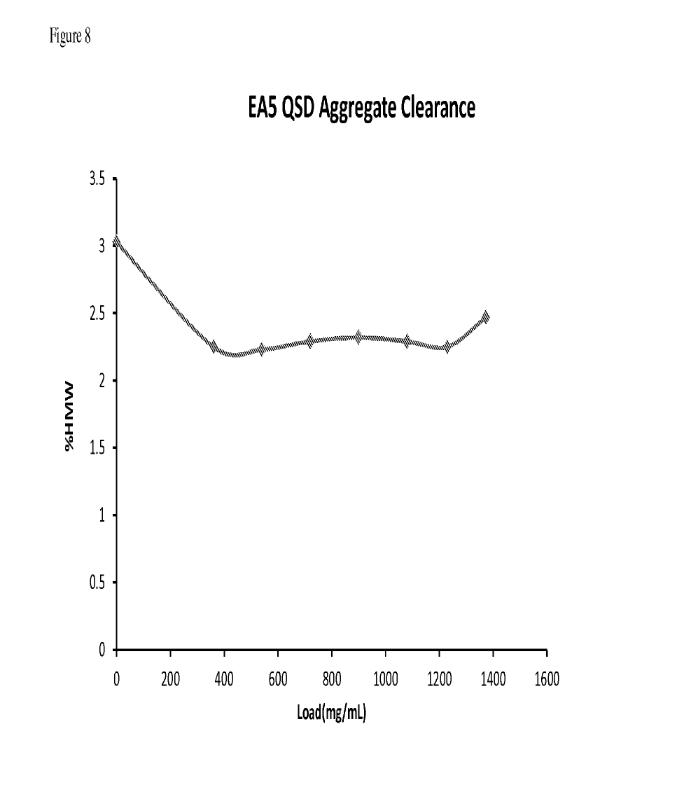

EA5 Protein A eluate (batch 93059BI) was conditioned to 4 mS/cm pH 6.5 and loaded onto the QSD. The results are shown in FIG. 8 and indicate minor aggregate reduction. HCP was not analyzed for this run. The step yield was 87%.

EA6

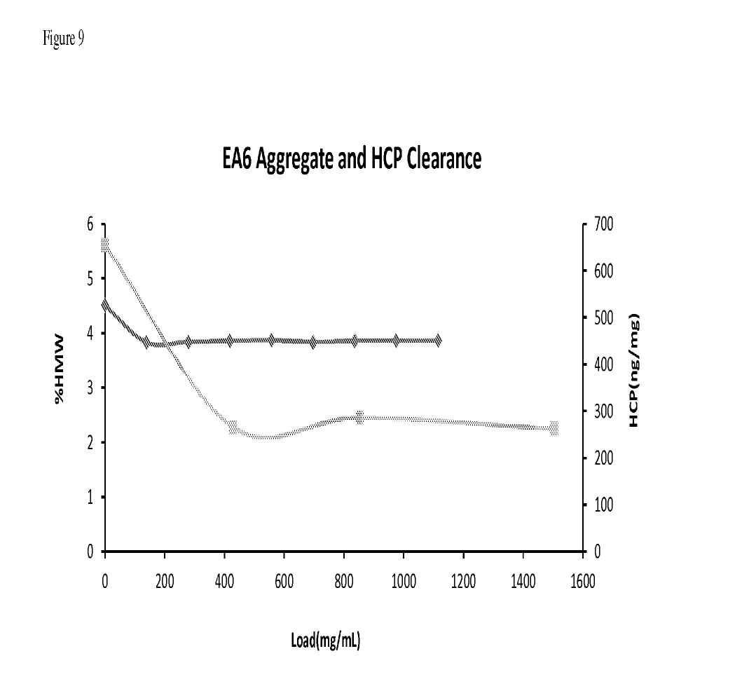

EA6 Protein A eluate (batch 1000020529) was conditioned to 4 mS/cm pH 6.5 and loaded onto the QSD. The results are shown in FIG. 9 where lines marked with diamonds represent aggregate reduction and lines marked with squares denote host cell protein (HCP) reduction. Minor aggregate reduction was observed. A 2-fold reduction in HCP content was also observed. The yield for this run was 99%.

EA7

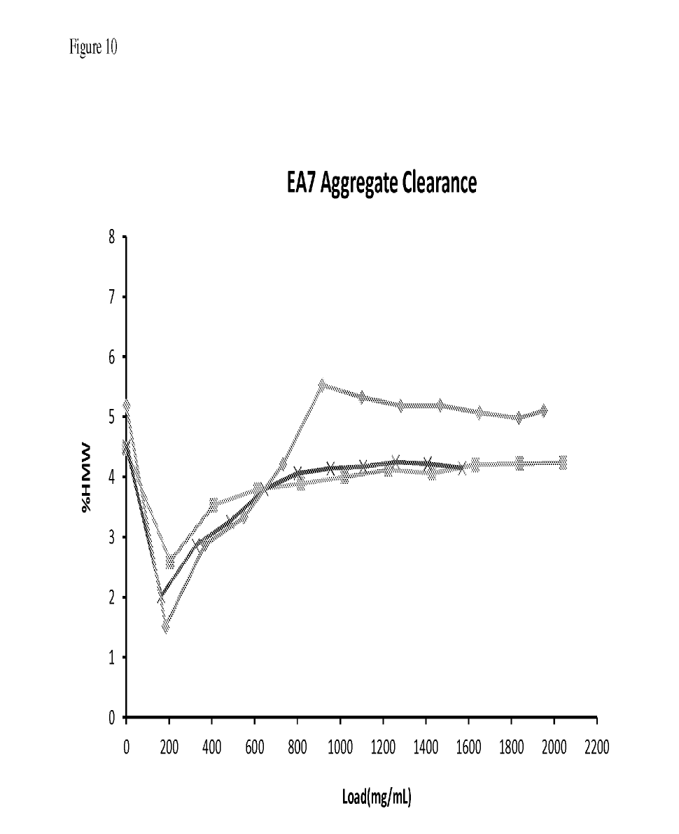

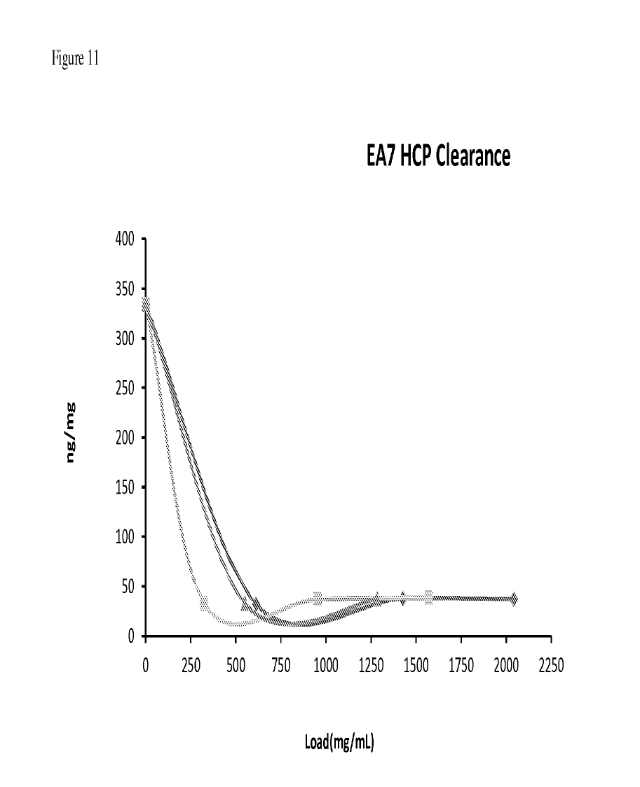

EA7 Protein A eluate (batch SUL091412) was conditioned to 4 mS/cm pH 5.5, pH 6.5, or 6 mS/cm pH 6.5 and loaded onto the QSD. The results for aggregate clearance are shown in FIG. 10 where an orange line denotes a conditioning at 4 mS/cm at pH5.5, a green line represents conditioning at 6 mS/cm at pH 6.5 and a blue line represents conditioning at 4 mS/cm at pH 6.5. EA7 alone shows good aggregate reduction at moderate loading levels (500 g/L) at all conditions tested. HCP clearance is shown in FIG. 11 where lines denoted in blue with diamonds represent HCP clearance at 4 mS/cm at pH5.5, lines in red with squares represent HCP clearance at 6 mS/cm at pH 6.5 and lines in green with triangles represent HCP clearance at 4 mS/cm at pH 6.5. These data are also consistent among runs (approx. 6-8 fold reduction i.e., 350 ng/mg to approximately 40 ng/mg). Yields ranged from 92-96%. As shown in FIG. 12, it is interesting to note the difference in aggregate clearance performance amongst the DVDs EA5 (blue with diamonds), EA6 (red with squares), and EA7 (blue with triangles), particularly between EA6 and EA7, which only differ by two amino acids.

### 2.2 EA1 Polishing by Sartobind Q Membrane Absorber

The DVD, EA1, was evaluated at bench scale for the ability of the Sartobind Q anion exchanger to remove aggregates and HCP. A Protein A eluate was clarified through a CE50 depth filter to remove turbidity, then conditioned to ˜5 mS/cm and pH 8.5, followed by loading over the Sartobind Q membrane which was pre-equilibrated with 20 mM Tris, 42 mM NaCl, pH 8.5 buffer. The feed concentration was 5-5.5 g/L, and the Q membrane was challenged to 1 to 3 kg/L membrane loading level at 1 ml/min flow rate. The flow-through pool along with the buffer wash fractions was collected and analyzed for levels of aggregates, monomer and HCP.

Table 6 summarizes the results from this experiment. Under these conditions significant aggregate reduction was observed. In addition, about 35-81% reduction in HCP content was obtained at the loading level of 1-3 kg/L. The recovery for this run was 96%.

TABLE 6
Sartobind Q Filtration of EA1

| Membrane | | | | | |
|---|---|---|---|---|---|
| loading | | Monomer | HWM | HCP | Yield |
| (kg/L) | Process Step | % | % | (ng/mg) | % |
| 1 | Load | 95.92 | 2.77 | 6408 | 101 |
| | FTW pool | 97.80 | 0.73 | 1240 | |
| 3 | Load | 95.69 | 2.87 | 8463 | 96 |
| | FTW pool | 98.10 | 0.67 | 5456 | |

### 2.3 EA1 Polishing by Mustang Q Membrane Absorber

The DVD, EA1, was evaluated with a bench scale-down model of Pall Mustang Q membrane for clearance of XMuLV and MMV virus at ambient temperature using a 1% (v/v) virus spike. Clearance of XMuLV and MVM was determined by infectivity assays. After equilibrating the device with 25 mM trolamine, 40 mM sodium chloride, pH 8.0 buffer, the membrane was challenged with spiked EA1 feed up to 1.3 kg/L membrane loading level followed by washing with the EQ buffer. The flow-through fractions and wash (FTW) pool were analyzed for virus titer. The process recovery was measured to be 102%. Overall, the Mustang Q membrane exhibited robust clearance of both XMuLV and MVM with minimum LRFs of 4.58±0.05 and 4.16±0.67, respectively.

Anion exchange chromatography can be selectively utilized to separate DVD-Igs from contaminating product (aggregates) and process impurities (HCP, virus). The unique properties associated with DVD molecules do not hinder the ability of the anion exchanger to bind the impurities while allowing the product of interest to flow through without significant loss.

### 3. Isolation and Purification of DVD-Igs Using Hydrophobic Interaction Chromatography

### 3.1 Phenyl Flow Through Polishing of DVD1

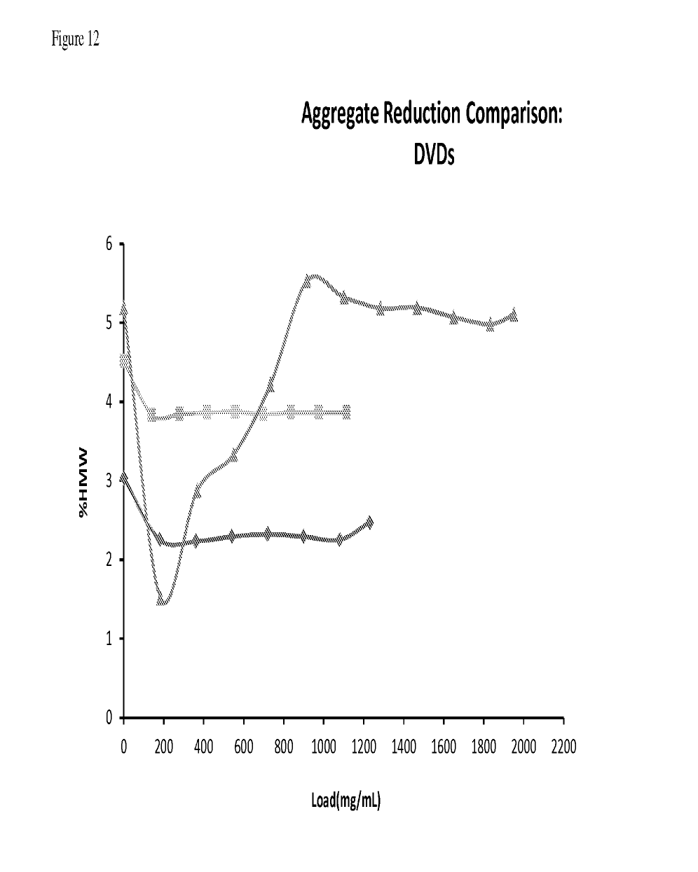

A bind and elute application of the Phenyl HP Sepharose resin was initially assessed to examine the clearance of the 20-25% aggregates contained in a Q FTW pool. FIG. 13 shows the elution and various regeneration conditions during Phenyl HP bind-elute processing for DVD1, an anti-TNF/PGE2 DVD-Ig. Bind and elute operation of the Phenyl HP column was able to reduce the aggregate levels to <1% for this DVD, but with only 40-50% recovery. From this experiment it was also observed that the high molecular weight species were very hydrophobic, with only half of the still bound material coming off of the column in the 25 mM sodium phosphate, 20% isopropyl alcohol regeneration step.

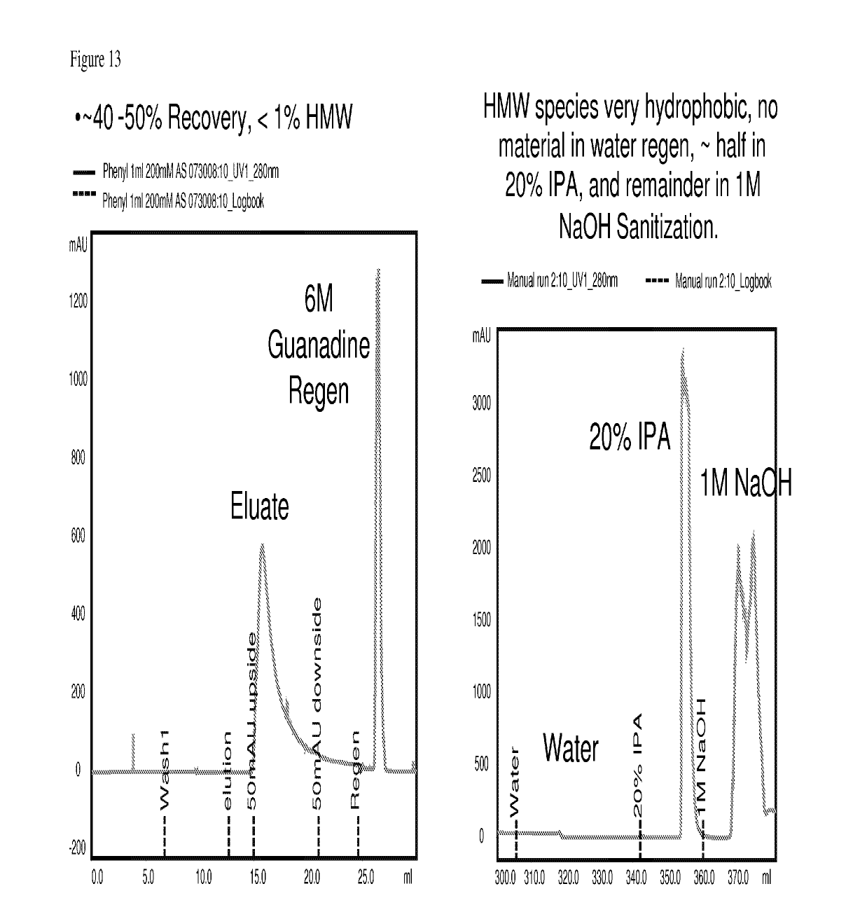

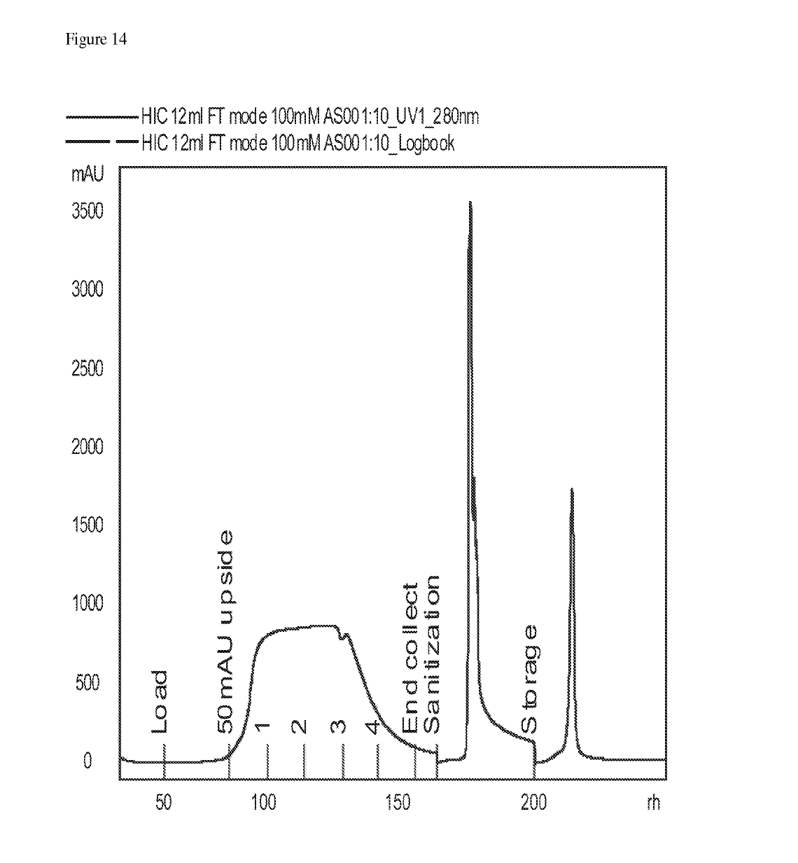

Various HIC resins including Octyl FF, Phenyl FF (High sub and Low sub), and Butyl S were evaluated for bind-elute purification of DVD 1. However, none of these resins provided a suitable level of aggregate clearance with acceptable product recovery. The flow through mode operation of the Phenyl HP column was then examined. A Q FTW sample pool was conditioned with 25 mM sodium phosphate, 1.7M ammonium sulfate to reach a final ammonium sulfate concentration of 100 mM. The column was then loaded up to 20 g/L. A 3 mM sodium phosphate, 100 mM ammonium sulfate buffer was used for equilibration and wash. A 25 mM sodium phosphate buffer containing 20% IPA was used for regeneration. WFI was used for rinse. 1M NaOH was used for sanitization and 0.1M NaOH was used for storage. FIG. 14 shows a representative flow through chromatogram for DVD1 under these conditions. Table 7 demonstrates the robustness of the flow-through Phenyl HP process through the examination of fractionation of the Phenyl FTW with varying ammonium sulfate concentrations. These results show the 100 mM ammonium sulfate condition balancing high product recovery with high product quality, with increases or decreases in ammonium sulfate concentration showing only minor variation in product quality and/or recovery, allowing for a relatively large operating window for this process. Altogether, <1% aggregates was achieved while increasing the recovery to 65-70%.

TABLE 7
DVD1 Product Quality and Recovery

| | % | % | | HCP |
|---|---|---|---|---|
| Sample | LMW | HMW | | (ng/mg) |
| 200 mM AS Phenyl Load | 0.19 | 11.4 | Yield = | |
| 200 mM AS FTW 1 | 0.68 | 0.18 | 57% | 312 |
| 200 mM AS FTW 2 | 0.19 | 0.12 | | 83 |
| 200 mM AS FTW 3 | 0.13 | 0.11 | | 55 |
| 200 mM AS FTW 4 | 0.13 | 0.14 | | 50 |
| 200 mM AS FTW 5 | 0.14 | 0.14 | | 58 |
| 200 mM AS FTW 6 | 0 | 0.21 | | 39 |
| 200 mM AS FTW 7 | 0 | 0.26 | | 9 |
| 150 mM AS Phenyl Load | 0.15 | 11.6 | Yield = | |
| 150 mM AS FTW 1 | 0.36 | 0.16 | 63% | 136 |
| 150 mM AS FTW 2 | 0.13 | 0.15 | | 46 |
| 150 mM AS FTW 3 | 0.13 | 0.22 | | 44 |
| 150 mM AS FTW 4 | 0 | 0.34 | | 29 |
| 150 mM AS FTW 5 | 0 | 0.42 | | 7 |
| 100 mM AS Phenyl Load | 0.19 | 11.6 | Yield = | |
| 100 mM AS FTW 1 | 0.25 | 0.16 | 70% | 79 |
| 100 mM AS FTW 2 | 0.12 | 0.21 | | 40 |
| 100 mM AS FTW 3 | 0.14 | 0.4 | | 41 |
| 100 mM AS FTW 4 | 0 | 0.63 | | 64 |
| 100 mM AS FTW 5 | 0.04 | 0.88 | | 9 |
| 50 mM AS Phenyl Load | 0.18 | 12.75 | Yield = | |
| 50 mM AS FTW 1 | 0.33 | 0.42 | 70% | 84 |
| 50 mM AS FTW 2 | 0.14 | 0.27 | | 38 |
| 50 mM AS FTW 3 | 0.13 | 0.47 | | 36 |
| 50 mM AS FTW 4 | 0.08 | 0.69 | | 40 |
| 50 mM AS FTW 5 | 0.03 | 0.99 | | 11 |
| 10 mM AS Phenyl Load | 0.18 | 12.81 | Yield = | |
| 10 mM AS FTW 1 | 0.25 | 0.38 | 54% | 75 |
| 10 mM AS FTW 2 | 0.09 | 0.35 | | 38 |
| 10 mM AS FTW 3 | 0.16 | 1.25 | | 36 |
| 10 mM AS FTW 4 | 0 | 0.38 | | 68 |

### 3.2 Phenyl Flow Through Polishing of DVD2

Phenyl FF (High Sub) resin was used for polishing DVD2, a DVD with high aggregation levels (15-20%). The identified processing conditions consisted of adding 50 mM sodium phosphate, 1.7 M ammonium sulfate buffer to the Q FTW to reach a final ammonium sulfate concentration of 140 mM. The column was then loaded up to 35 g/L. The solutions used were 50 mM sodium phosphate, 140 mM ammonium sulfate for equilibration and wash, 25 mM sodium phosphate, 20% IPA for regeneration, a WFI rinse, sanitization with 0.5M NaOH and storage in 0.1M NaOH. FIG. 15 shows a representative flow through chromatogram for DVD2 under these conditions. Aggregates were reduced from 17% down to 1.1%, with a product recovery of 66%. Product recovery for the three 3000 L GMP batches resulted in a product recovery range of 56-62%, with final BDS levels of aggregates being <0.5%.

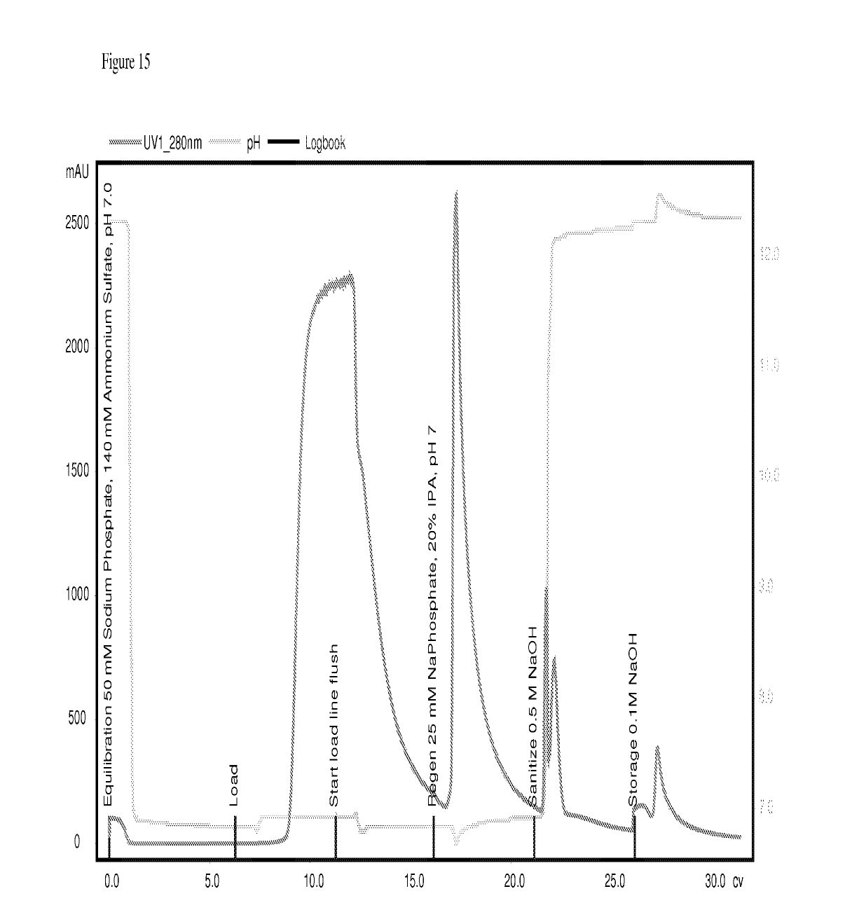

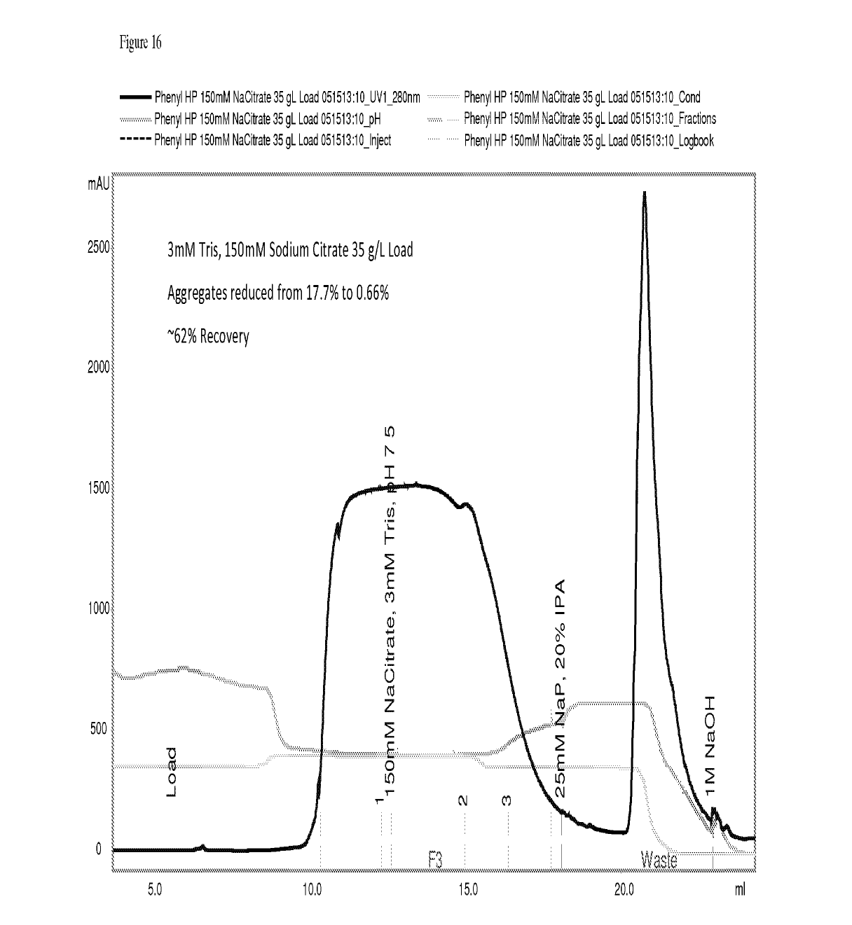

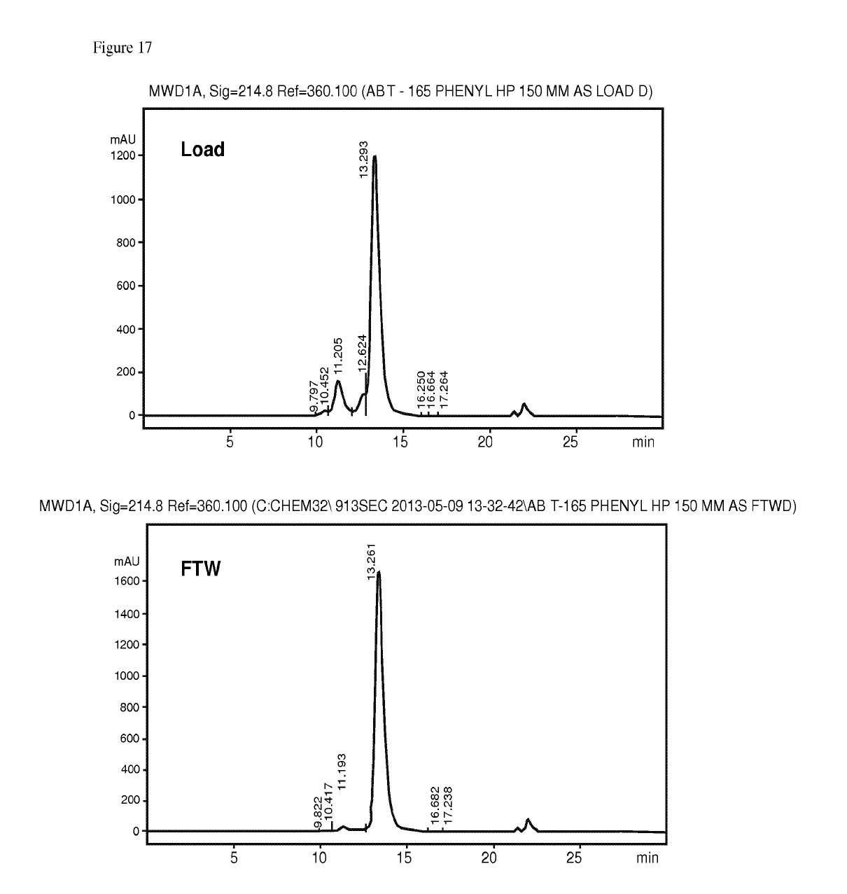

In addition to the ammonium sulfate buffer system used in the above examples, sodium citrate based buffer system was also evaluated for DVD2. FIG. 16 shows the DVD2 Phenyl HP flow through process using 150 mM sodium citrate buffer. This process was able to reduce aggregate levels from ˜17.7% down to ˜0.7%, with 62% recovery. Note that comparable product throughput, recovery and quality were obtained when using Phenyl Sepharose HP resin versus the Phenyl FF (High Sub) resin for flow-through polishing. Table 8 summarizes the effects of varying ammonium sulfate or sodium citrate concentration on product recovery and product quality by Phenyl HP flow-through polishing. FIG. 17 show representative SEC chromatograms for DVD2 Phenyl Load and FTW samples.

TABLE 8
DVD2 Product Quality and Recovery

| | | % | % | % | % |
|---|---|---|---|---|---|
| | % | HMW | LMW | HMW | LMW |
| Condition | Recovery | Load | Load | FTW | FTW |
| 100 mM Ammonium | 70 | 17.78 | 0.73 | 7.09 | 2.11 |
| Sulfate (15 g/L) | | | | | |
| 150 mM Ammonium | 62 | 17.63 | 0.67 | 1.37 | 0.71 |
| Sulfate (15 g/L) | | | | | |
| 50 mM Sodium | 72 | 17.73 | 0.76 | 7.76 | 0.52 |
| Citrate (15 g/L) | | | | | |
| 100 mM Sodium | 62 | 17.83 | 0.69 | 0.88 | 0.51 |
| Citrate (15 g/L) | | | | | |
| 150 mM Sodium | 62 | 17.69 | 0.68 | 0.66 | 1.90 |
| Citrate (35 g/L) | | | | | |

### 3.3 Phenyl Flow Through Polishing of DVD3

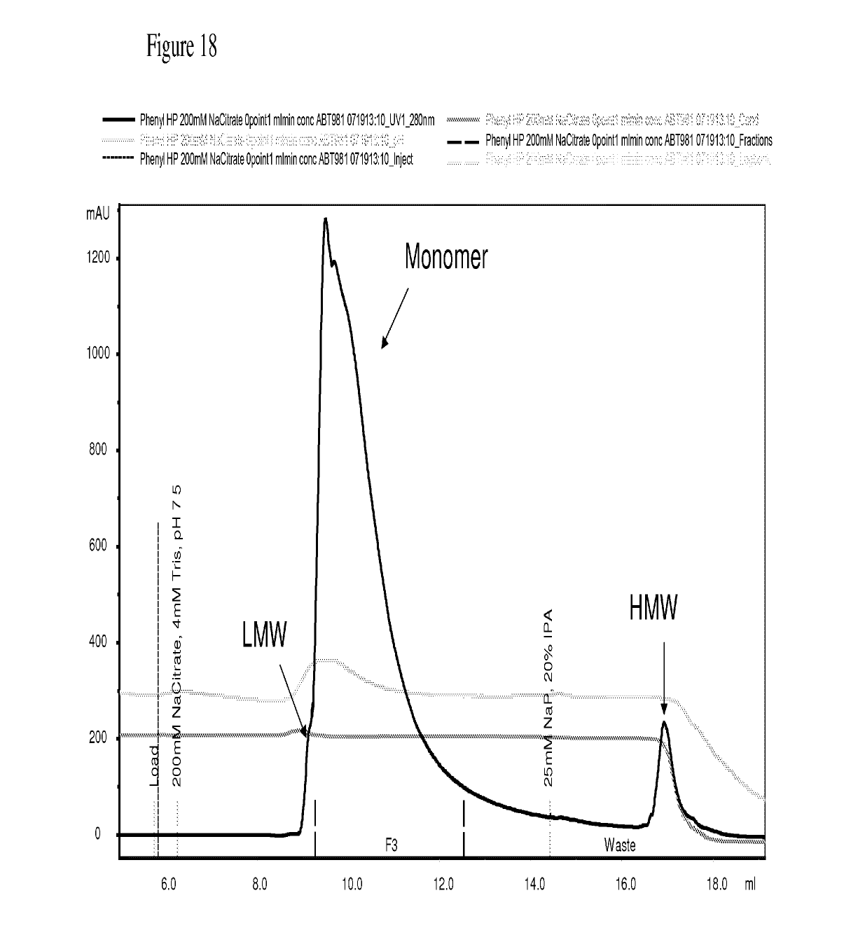

With the successful adaptation of a Phenyl HP flow through process for DVD2, the process was applied to DVD3, an anti-IL-1α/IL-1β DVD. DVD3 is a low aggregation DVD molecule (typically 1.5-3.5%), and also contains 1-2% of fragments. Table 9 shows the results of varying the sodium citrate levels on the amount of aggregation and fragmentation observed during Phenyl HP flow-through evaluations. The co-elution of the fragments on the front of the flow-through wash peak reduced the ability to drastically reduce the fragmentation levels in the FTW. To minimize the load volume, thus increasing the resolution between the fragments and monomer, Q FTW material was concentrated by approximately 7 fold. Table 10 shows the results of varying the sodium citrate levels on the amount of aggregation and fragmentation observed during Phenyl HP flow-through evaluations utilizing concentrated Q FTW material. FIG. 18 shows the chromatographic profile for a DVD-Ig™ utilizing 200 mM sodium citrate, showing the relative location of the fragments, monomer and aggregates. This process was able to reduce aggregates from 3.5% to 0.7% and fragments from 1.8% to 1.1% with 84% recovery.

TABLE 9
DVD3 Product Quality and Recovery

| | | % | % | % | % |
|---|---|---|---|---|---|
| | % | HMW | LMW | HMW | LMW |
| Condition | Recovery | Load | Load | FTW | FTW |
| 300 mM Sodium | 36 | 1.74 | 2.41 | 0.4 | 5.64 |
| Citrate | | | | | |
| 200 mM Sodium | 73 | 1.40 | 1.68 | 0.46 | 1.37 |
| Citrate | | | | | |
| 100 mM Sodium | 78 | 1.18 | 1.63 | 0.98 | 1.24 |
| Citrate | | | | | |
| 150 mM Sodium | 88 | 1.49 | 1.67 | 0.93 | 1.25 |
| Citrate | | | | | |

TABLE 10
DVD3 Product Quality and Recovery (Post-Concentration)

| | | % | % | % | % |
|---|---|---|---|---|---|
| | % | HMW | LMW | HMW | LMW |
| Condition | Recovery | Load | Load | FTW | FTW |
| 150 mM Sodium | 43 | 2.36 | 1.69 | 1.42 | 1.16 |
| Citrate | | | | | |
| 250 mM Sodium | 67 | 6.44 | 1.34 | 0.31 | 0.95 |
| Citrate | | | | | |
| 200 mM Sodium | 84 | 3.52 | 1.79 | 0.7 | 1.07 |
| Citrate | | | | | |

### 4. Filtration of DVD-Ig™ Molecules

### 4.1 Filtration Methods

Three purified DVD-Ig™ feed streams, DVD-1, DVD-2, and DVD-3, were evaluated for viral filtration performance (flux, flux decay, and throughput) using commercially available viral filters at bench scale. A Q Sepharose® flow-through eluate pool of each DVD was used as the filtration feed and the specific conditions (feed stream DVD concentration, pH, and conductivity) are shown in Table 11. The filters used in this study are listed in Table 12. Each filter was flushed with the appropriate buffer at the respective feed pH (e.g. 50 mM NaAc, pH 5; or 25 mM trolamine, 40 mM NaCl, pH 8) before feed loading. The feed was 0.1 μm filtered at a loading level in the range of 360-405 L/m2. All experiments were run under constant pressures as shown in Table 12. The flux rate and volume were recorded throughout the runs until the targeted flux decay (90%) was reached or the available feed was depleted. The product recovery was determined by measuring the filtrate pool concentration and total volume.

TABLE 11
DVD-Ig ™ feed used for viral filtration study

| | Concentration | | Conductivity |
|---|---|---|---|
| Feed | (g/L) | pH | (ms/cm) |
| DVD-1 | 2.2-2.3 | 5, 8.2 | 3.8-4.6 |
| | 9.6-9.8 | 5, 8 | 4-4.7 |
| DVD-2 | 3.0 | 5, 6.5 | 4.1-4.2 |
| DVD-3 | 7.7-7.8 | 5, 6.8 | 3.9-4.0 |

TABLE 12
Viral filtration experimental condition

| | | Area | Constant Pressure |
|---|---|---|---|
| Filters | Membranes | (cm²) | (psi) |
| Virosart Mini | PES | 5 | 30 |
| Viresolve Pro | PES | 3 | 30 |
| Ultipor ® VF | PVDF | 9.6 | 30 |
| DV20 | | | |
| Planova 20N | Cellulose | 10 | 14 |
| Planova BioEx | PVDF | 3 | 42 |

### 4.2 Filtration Analysis

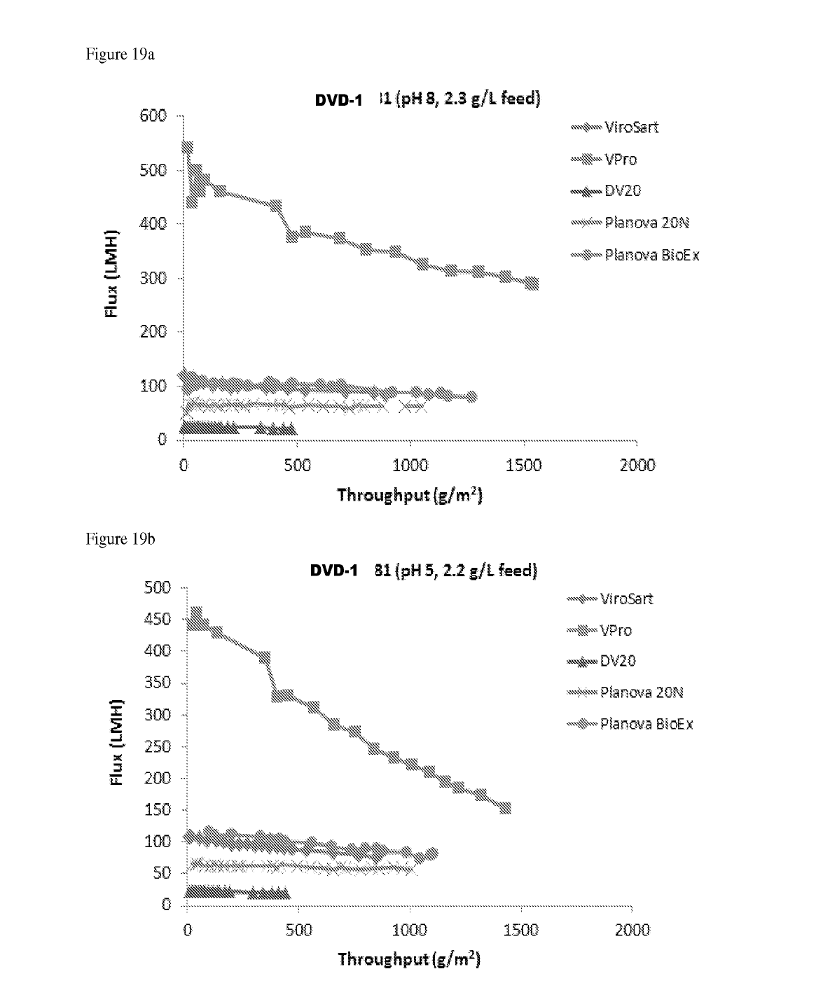

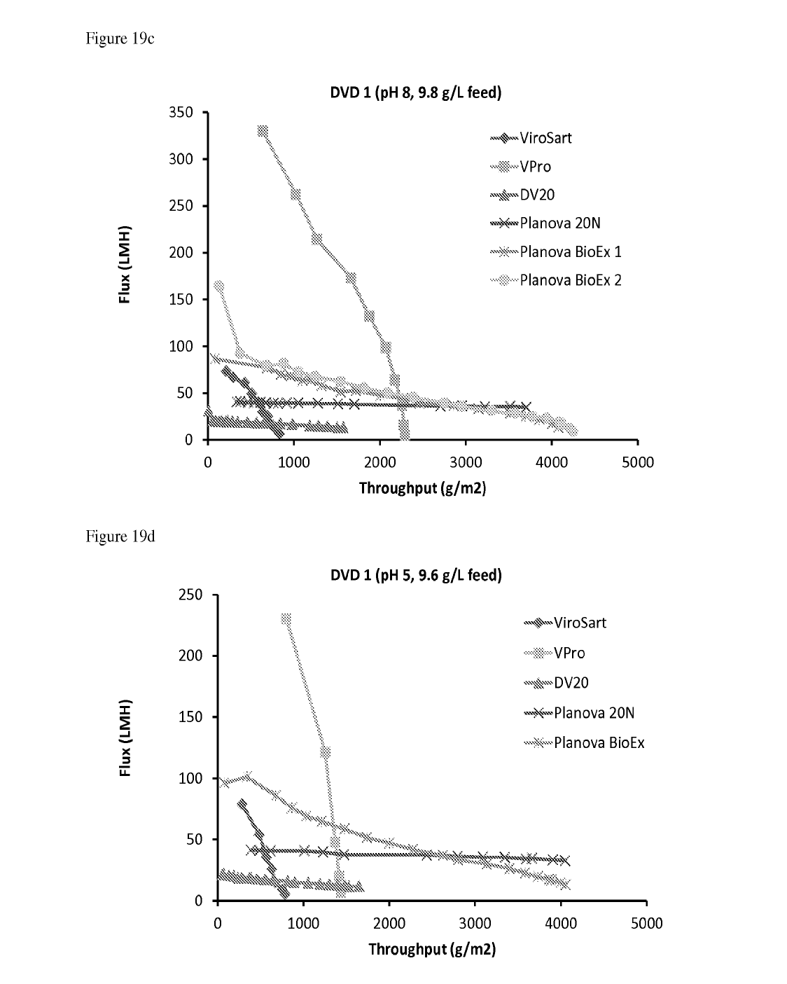

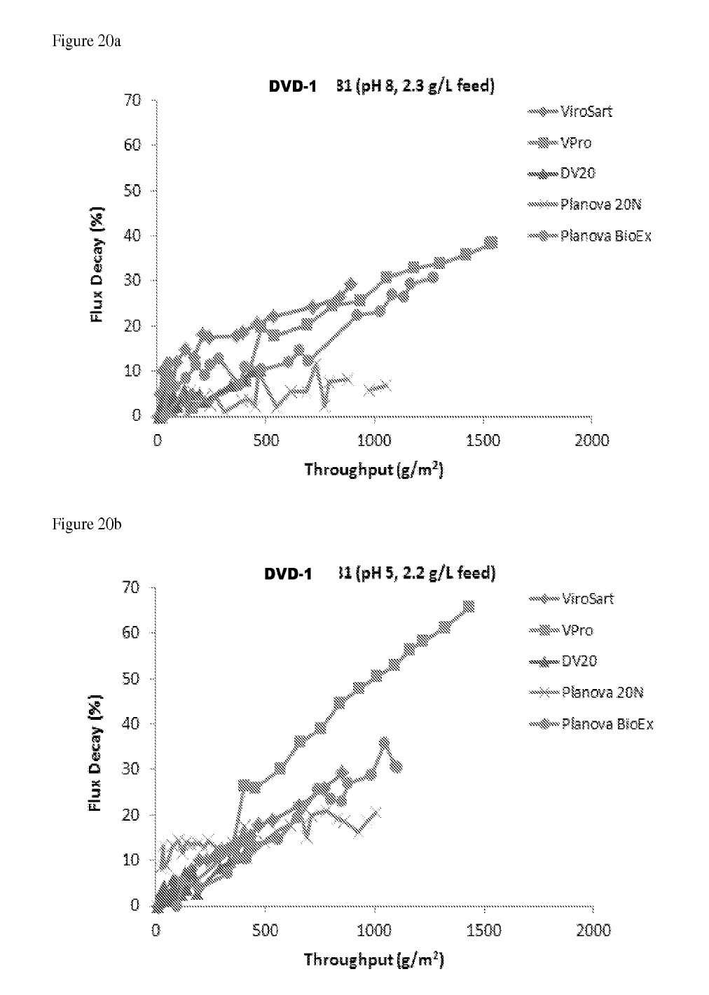

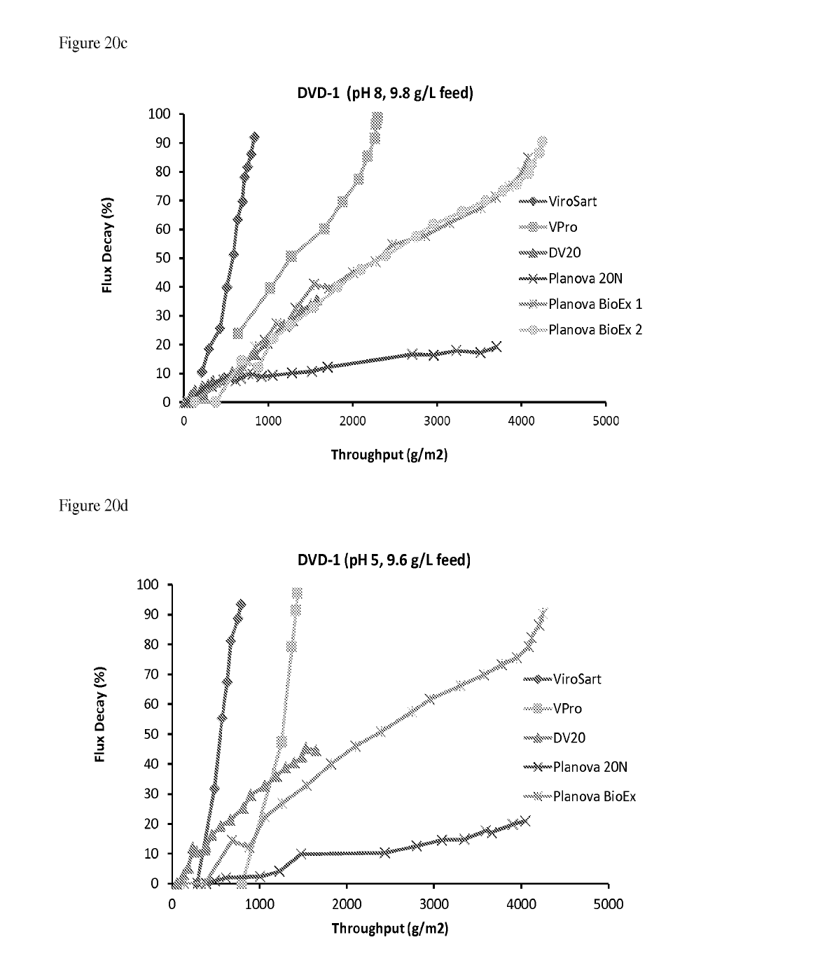

FIGS. 19a and 19b show the fluxes as a function of throughput for each filter when processing a low concentration (2.2-2.3 g/L) DVD-1 feed at pH 8.2 and 5, respectively. Viresolve Pro (VPro) showed the highest flux among all the filters. FIGS. 19c and 19d showed the filter throughput performance when processing a high concentration (9.8 g/L) DVD-1 feed at pH 8 and 5, respectively. Although the VPro showed the highest initial flux, as shown in FIGS. 20(a-d), the flux decay for this filter was also among the highest at the examined concentration and pH conditions. ViroSart and Planova™ BioEx showed similar flux profiles at both pHs for the low protein concentration feed, but the latter appeared to give lower flux decay than the former. At elevated protein concentrations, such difference is amplified and Planova™ BioEx showed significantly higher flux than Virosart. Planova 20N and DV20 gave the lowest flux rates but also the least flux decays. The final throughput achieved by these filters ranged from about 500 to 4000 g/m2, mostly due to feed availability or processing time constraints.

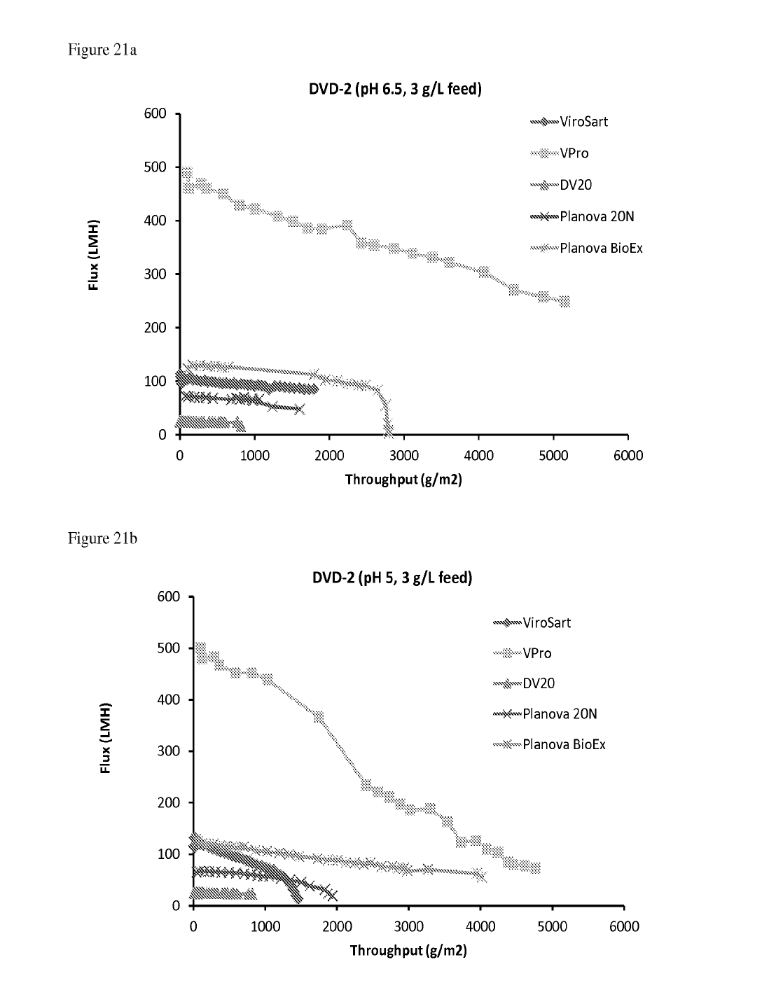

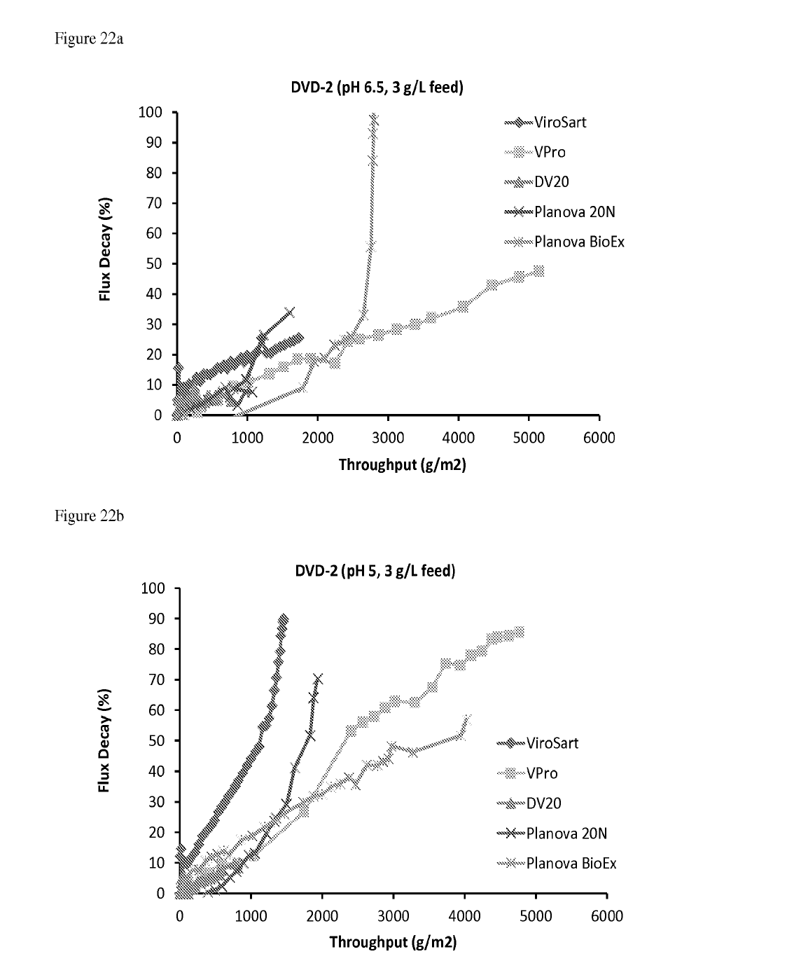

FIGS. 21a and 21b show the fluxes as a function of throughput for each filter when processing a low concentration (˜3 g/L) DVD-2 feed at pH 6.5 and 5, respectively. At both pH conditions, all the filters achieved >1000 g/m2 throughput. Interestingly, FIGS. 22a and 22b demonstrate that the effect of pH on flux-throughput performance varied among filters; lower pH seems to increase flux decay for VPro and ViroSart but to decrease the flux decay for Planova™ BioEx. For this feed stream, a throughput of 4-5 kg/m2 was achieved at either pH condition.

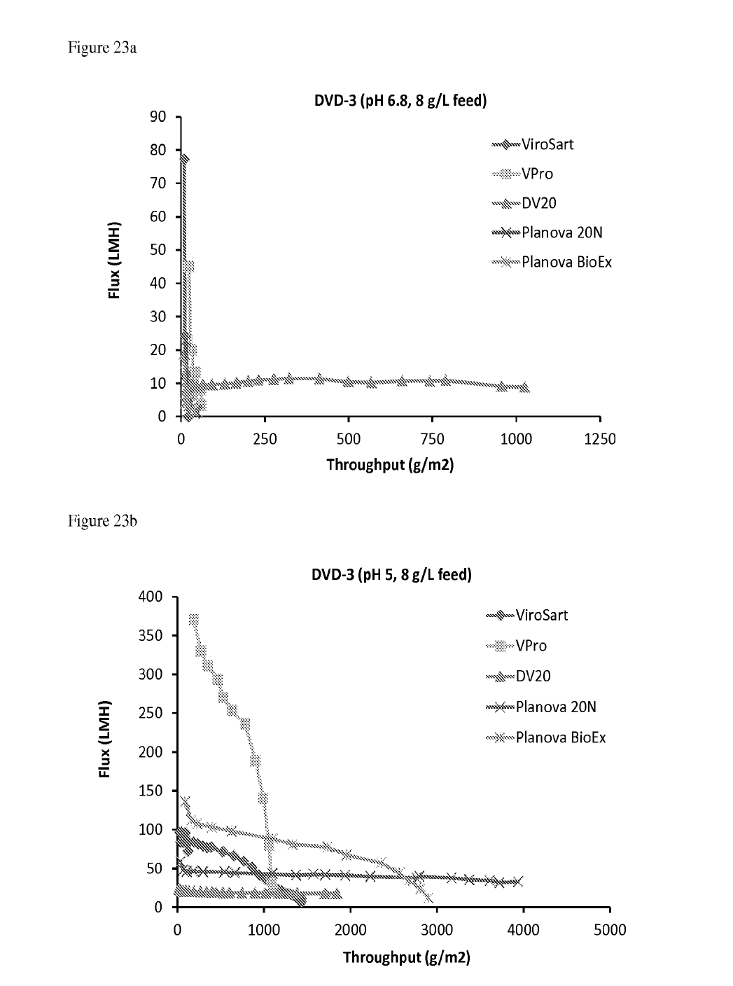

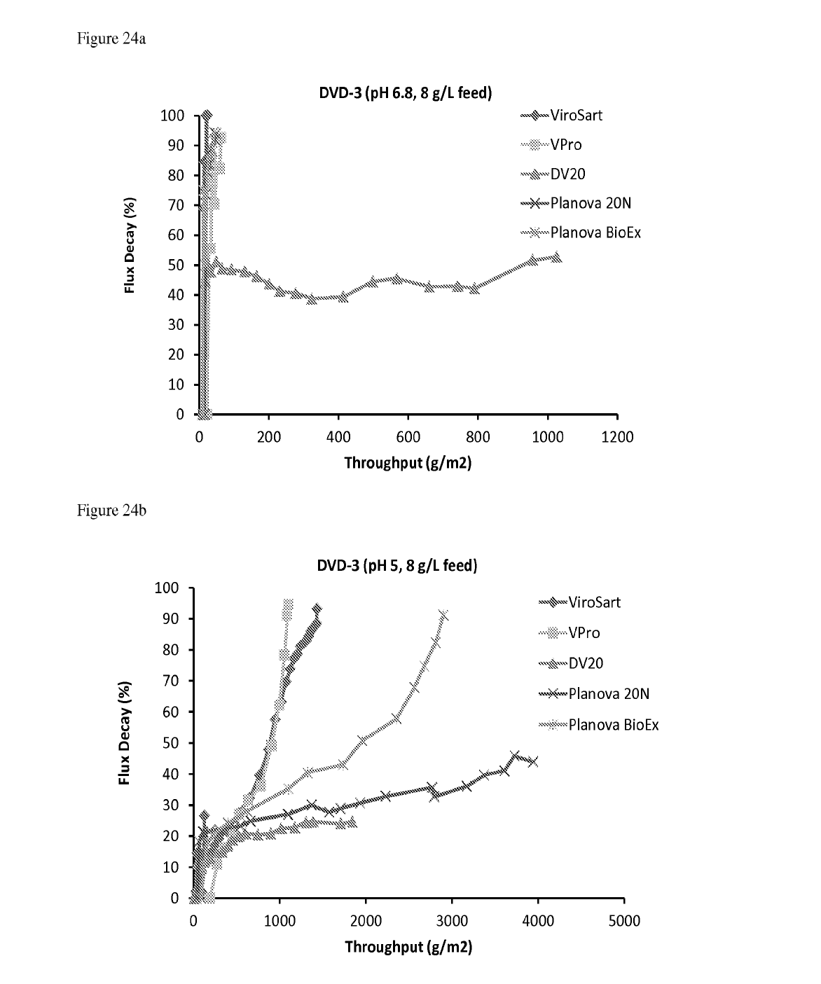

FIGS. 23a and 23b show the fluxes as a function of throughput for each filter when processing a high concentration (˜8 g/L) DVD-3 feed at pH 6.8 and 5, respectively. As shown in FIG. 23a, at pH 6.8, all filters clogged shortly after the run starts with the exception of DV20. As shown in FIG. 23b, at pH 5, all filter flux performances were significantly improved, with Planova™ BioEx and 20N reaching 2.9 and 3.9 kg/m2, respectively, at the end of the run. Although its flux rate was very low (˜10 LMH), FIG. 24a shows that DV20 can be challenged to 1 kg/m2 without showing further flux decay. FIG. 24b shows that the Planova 20N can be challenged further as its flux decay was below 50%. VPro and ViroSart showed the greatest flux decay but achieved 1.1 and 1.4 kg/m2 loading at the end of processing. Again DV20 showed the least flux decay among all the tested filters with a throughput of at least 1.8 kg/m2.

Table 13 summarizes the product yields for the 3 DVD feed streams from all the filtration runs. For the DVD-1 and DVD-2 feed streams, ≧97% product recovery was observed at the pHs and feed stream concentrations tested. For the DVD-3 high concentration (˜8 g/L) feed stream at pH 6.8, significant product loss was seen for the BioEx, Planova 20N, and Virosart CPV filters, while all of the filters showed ≧99% product recovery at pH 5.

In summary, despite their large effective size and variable molecular conformations, these surprising data demonstrate that DVD-Igs can be processed using standard viral filters. BioEx, Planova 20N, and DV20 filters had lower flux decays compared to ViroSart and VPro, though the latter may have better fluxes. Since viral filter loading is limited by the level of flux decay, the hydrophilic PVDF- or cellulose-based viral filters may be preferred for DVD processing in order to provide more consistent performances. Finally, when feed streams contain higher concentrations of DVDs, reducing feed pH may improve filter throughput performance.

TABLE 13
Viral filtration step yield DVD feed streams

| | DVD-1 | | DVD-2 | | DVD-3 | |
|---|---|---|---|---|---|---|
| | Yield (%) | | Yield (%) | | Yield (%) | |
| Filters | pH 5 | pH 8 | pH 5 | pH 6.5 | pH 5 | pH 6.8 |
| Planova | 101 | 97 | 100 | N.D. | 101 | 40 |
| BioEx | | | | | | |
| Planova 20N | 98 | 101 | 97 | 97 | 100 | 22 |
| Ultipor | 102 | 99 | 100 | 103 | 99 | 98 |
| DV20 | | | | | | |
| Viresolve | 98 | 102 | 101 | 103 | 99 | 98 |
| Pro | | | | | | |
| Virosart CPV | 100 | 98 | 101 | 103 | 101 | 40 |

### 4.3 Hydrophobic Analysis of DVD-Ig™ Molecules

The hydrophobicity of the above three DVDs along with other DVD molecules and various mAbs were measured using an analytical HIC method. Specifically, a TSKgel butyl NPR column (2.5 um, 4.6 mm i.d.×3.5 cm length) was used and run at 30° C. The mobile phase A was 20 mM Tris, 1.5M ammonium sulfate pH 7.0 buffer, and mobile phase B was 20 mM Tris, pH 7 buffer. After equilibration, 25 μg of each protein was injected into the column and a linear gradient from buffer A to buffer B was run following the scheme defined in Table 14. The flow rate was 1 ml/min with a total run time of 45 min. The UV absorbance of the elution profile was monitored at 214 nm, from which the retention time corresponding to the peak apex and the half-height peak width were determined.

TABLE 14
Analytical HIC method mobile phase run scheme

| Time | % |
|---|---|
| (min) | B |
| 0 | 0% |
| 2 | 0% |
| 32 | 100% |
| 37 | 100% |
| 39 | 0% |
| 45 | stop |

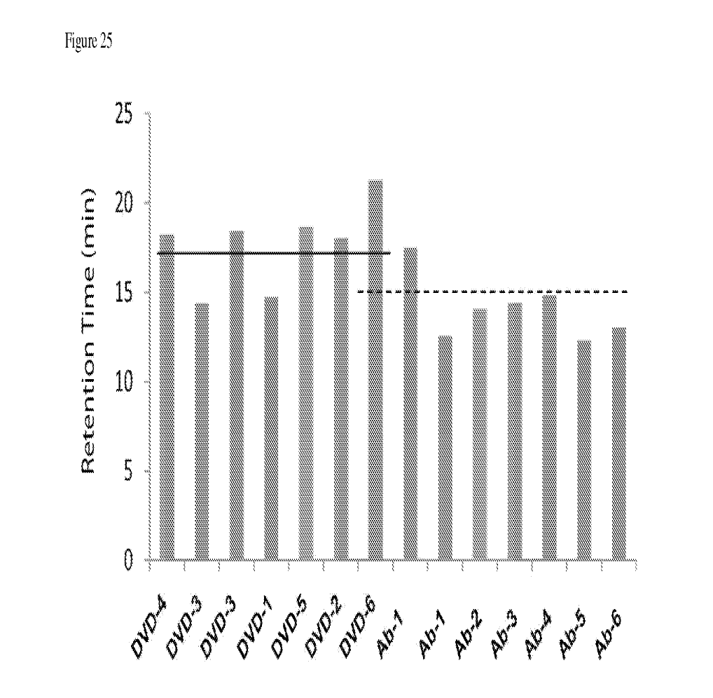

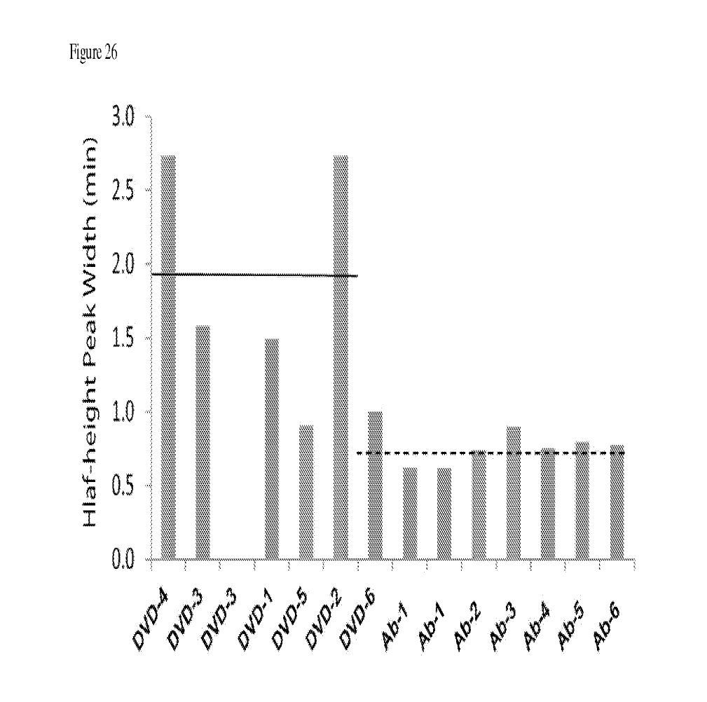

The retention time of each molecule and the peak width (at half height of the peak) can be determined from the HIC elution profile for each molecule, as summarized in FIGS. 25 and 26. In general, the longer the retention time and the wider the elution peak then the stronger binding of the protein to the resin, in this case by hydrophobic interaction. Hence, by comparing these values one can assess the relative hydrophobicity of the proteins of interest. In FIGS. 25 and 26, the solid line represents the average retention time or peak width for all the DVDs tested while the dashed line denotes the average value for all the mAbs (also shown in Table 15). The majority of tested DVDs showed longer retention times and wider elution peaks than the mAbs, indicating that this class of molecules is significantly more hydrophobic.

Linking this molecular characteristic to the observed viral filtration performance, it seems that the hydrophilized PVDF- or cellulose-based viral filter (such as Planova™ BioEx, 20N or DV20) may be less hydrophobic, and as a consequence less amenable to protein binding via hydrophobic interaction. Thus, these filters may reduce protein binding, thereby alleviating membrane fouling and reduce the extent of flux decay during filtration.

TABLE 15
Average retention time and half-
height peak width for DVDs vs. mAbs

| | Retention time | Peak width at half-height |
|---|---|---|
| Molecules | (min) | (min) |
| DVD-Ig | 17.1 | 1.9 |
| mAb | 15.0 | 0.8 |

### 4.4 Viral Clearance by DVD-Ig™ Filtration

The performance of virus reduction by DV20 or Virosart CPV filter was measured with DVD 1 and 3 using XMuLV and MMV. Both sets of experiments were performed at constant pressure of 30 psi. The feed protein concentration was 3.2 g/L for DVD 1 and 2.8 g/L for DVD 3, and pH was 8 and 5.5 for DVD 1 and DVD 3, respectively. The filter was challenged to 386 to 1400 g/m². Table 16 summarizes the log reduction values (LRV) for both viruses with the two DVD feed streams. Under examined conditions, ≧3 log reduction of both viruses was observed for the two DVD feedstream by different filters.

TABLE 16
Virus clearance LRV during DVD-Ig ™ filtration

| DVDs | Filter | Conditions | XMuLV | MVM |
|---|---|---|---|---|
| DVD-1 | DV 20 | pH 8, 3.2 g/L, | ≧5.4 | ≧2.97 |
| | | 386 g/m² loading | | |
| DVD-2 | Planova | pH 6.5, 3.2 g/L, | ≧5.99 | 3.93 |
| | 20N | 954 g/m2 loading | | |
| DVD-3 | Virosart | pH 5.5, 2.8 g/L, | ≧3.19 | ≧6.27 |
| | CPV | 1400 g/m² loading | | |

Various publications are cited herein, the contents of which are hereby incorporated by reference in their entireties.

What is claimed is:

1. A method for producing a product- or process-related impurity reduced DVD-Ig preparation from a load sample mixture comprising a DVD-Ig and at least one product- or process-related impurity said method comprising the steps of:
(a) contacting said load sample to a mixed mode resin; and
(b) collecting a product sample, wherein
said product sample comprises said product- or process-related impurity-reduced DVD-Ig preparation.

2. The method of claim 1, wherein said mixed mode resin consists of an anionic charge or has a cation exchange functionality.

3. The method of claim 1, wherein contacting said load sample mixture with the mixed mode resin is performed in a flow through mode.

4. The method of claim 1, wherein contacting said load sample mixture with the mixed mode resin is performed in a batch adsorption mode.

5. The method of claim 2 wherein said resin is selected from the group consisting of Capto MMC, Capto MMC ImpRes, Nuvia cPrime, and Toyopearl MX Trp-650M

6. The method of claim 1, wherein the pH of said equilibration buffer and said load sample mixture is about 1 to 4 pH units lower than the pI of the protein of interest.

7. The method of claim 1, wherein the conductivity of said equilibration buffer and said sample is about 2 to 20 mS/cm.

8. The method of claim 1, wherein the resin loading level of said mixed mode resin is about 200 to about 1200 g/L.

9. The method of claim 1, wherein the product sample comprises reduced level of product- and/or process-related impurities than the said load sample mixture.

10. The method of claim 9, wherein the said product-related impurities are DVD-Ig aggregates and fragments, and the process-related impurities are HCPs.

11. The method of claim 1, wherein the said load sample mixture is obtained from unit operations consisting of at least one chromatography step.

12. The method of claim 11, wherein the chromatography step is an affinity chromatography, an ion exchange chromatography, and/or another mixed mode chromatography.

13. The method of claim 12, wherein the affinity chromatography step is a Protein A chromatography.

14. The method of claim 12, wherein the ion exchange chromatography step is an anion exchange chromatography.

15. The method of claim 14, wherein the anion exchanger chromatography is running in flow-through mode.

16. The method of claim 15, wherein the anion exchanger is a Q membrane adsorber.

17. The method of claim 16, wherein the Q membrane adsorber is selected from the group consisting of Sartobind Q membrane, Mustang Q membrane, Qyuspeed Q membrane, and Sartobind STIC membrane adsorber.

18. The method of claim 12, wherein the said another mixed mode chromatography step is an anion exchanger-based mixed mode chromatography.

19. The method of claim 18, wherein the anion exchanger-based mixed mode chromatography is operating in flow-through mode.

20. The method of claim 19, wherein the anion exchanger-based mixed mode resin is selected from the group consisting of Capto Adhere and Capto Adhere ImpRes.

21. The method of claim 1, wherein the said product sample is further purified through another chromatography step.

22. The method of claim 21, wherein the said another chromatography step is an ion exchange chromatography step, or another mixed mode chromatography step.

23. A method for producing a product- or process-related impurity-reduced DVD-Ig preparation from a load sample mixture comprising the protein of interest and at least one product- or process-related impurities, said method comprising the steps of:
(a) subjecting the said load sample mixture to Protein A chromatography step to obtain an Protein A eluate sample;
(b) contacting said Protein A eluate sample to a cation exchanger based mixed mode resin and collecting the flow-through pool to obtain a cation-exchanger based mixed mode eluate sample; and
(c) subjecting the said mixed mode eluate sample to a second chromatography step to obtain a final sample, wherein
the said final sample comprises impurity-reduced protein preparation.

24. The method of claim 23, wherein the said second chromatography step is selected from a group consisting of anion exchange and anion-exchanger based mixed mode chromatography.

25. A method for producing a product- or process-related impurity-reduced DVD-Ig preparation from a load sample mixture comprising the protein of interest and at least one product- or process-related impurities, said method comprising the steps of:
(a) subjecting the said load sample mixture to Protein A chromatography step to obtain an Protein A eluate sample;
(b) contacting said Protein A eluate sample to an anion exchange chromatography to obtain an AEX eluate sample; and
(c) contacting said AEX eluate sample to a cation exchanger based mixed mode resin and collecting the flow-through pool to obtain a final sample, wherein
the said final sample comprises impurity-reduced protein preparation.

26. A method for producing a product- or process-related impurity reduced DVD-Ig preparation from a load sample mixture comprising the protein of interest and at least one product- or process-related impurities, said method comprising the steps of:
(a) subjecting the said load sample mixture to Protein A chromatography step to obtain an Protein A eluate sample; and
(b) contacting said Protein A eluate sample to an anion exchange chromatography to obtain an AEX eluate sample; and
(c) contacting said AEX eluate sample to an anion exchanger-based mixed mode chromatography to obtain an AEX-MM eluate sample; and
(d) contacting said AEX-MM eluate sample to a cation exchanger based mixed mode resin and collecting the flow-through pool to obtain a final sample, wherein
the said final sample comprises impurity-reduced protein preparation.

27. The method of any one of claims 1-26, wherein the said protein is a DVD-Ig.

28. As a composition of matter, a DVD-Ig preparation produced by the method of claim 1.

29. As a composition of matter, a DVD-Ig preparation produced by any of the methods of claims 2-26.

30. A method for producing a product- or process-related impurity reduced DVD-Ig preparation from a sample mixture comprising an DVD-Ig and at least one product- or process-related impurity said method comprising the steps of:
(a) contacting said sample to an anion exchange resin or membrane absorber; and
(b) collecting a final sample, wherein
said final sample comprises said product- or process-related impurity-reduced DVD-Ig preparation.

31. The method of claim 30, wherein said anion exchange is performed in a flow through mode.

32. The method of claim 30 wherein said membrane absorber is selected from the group consisting of QyuSpeed D(QSD), Mustang Q, Sartobind Q, and Sartobind STIC membrane absorbers.

33. As a composition of matter, a DVD-Ig preparation produced by the method of any one of claims 30-32.

34. A method for producing a DVD-Ig preparation from a sample mixture comprising a DVD-Ig and a viral particle, wherein the preparation comprises a decreased number of viral particles or decreased viral activity in comparison to the sample mixture, the method comprising the steps of:
(a) applying the sample mixture to a first end of a nanofilter, the nanofilter comprising a nominal pore size of 20 nm;
(b) applying a constant pressure to the first end of the nanofilter; and
(c) collecting the DVD-Ig preparation from a second end of the nanofilter.

35. The method of claim 34, wherein the nanofilter comprises a material selected from the group consisting of polyestersulfone (PES), polyvinylidene fluoride (PVDF), and cellulose.

36. The method of claim 35, wherein the nanofilter is selected from the group consisting of Zeta Plus VR, Virosart CPV, Virosart HC, Virosart HF, Viresolve Pro, Ultipor VF DV20, Planova 20N. and Planova BioEx.

37. The method of claim 34, wherein the conductivity of the sample mixture is about 2 to about 12 S/mmS/cm.

38. The method of claim 34, wherein the concentration of the DVD-Ig in the sample mixture is about 2 to about 10 g/L.

39. The method of claim 34, wherein the DVD-Ig in the sample mixture and/or DVD-Ig preparation has a retention time on a hydrophobic interaction chromatography (HIC) column of about 13 to about 22.5 min.

40. The method of claim 34, wherein the DVD-Ig in the sample mixture and/or DVD-Ig preparation has a greater average retention time than a monoclonal antibody or antigen binding fragment thereof, and optionally, a greater average retention time than the monoclonal antibody or antigen binding fragment thereof comprising at least one antigen binding domain of the DVD-Ig.

41. The method of claim 34, wherein the DVD-Ig in the sample mixture and/or DVD-Ig preparation has a HIC elution profile half-height peak width of about 0.8 to about 2.7 min.

42. The method of claim 34, wherein the DVD-Ig in the sample mixture and/or DVD-Ig preparation has a greater HIC elution profile half-height peak width than a monoclonal antibody or antigen binding fragment thereof, and optionally, a greater average retention time than the monoclonal antibody or antigen binding fragment thereof comprising at least one antigen binding domain of the DVD-Ig.

43. The method of claim 34, wherein the pH of the sample mixture is about 5.0 to about 8.2.

44. The method of claim 34, wherein the pressure applied to the first end of the sample mixture is about 14 to about 42 psi.

44. The method of any one of claims 34-43, wherein
(a) the flux through the nanofilter is about 0 to about 550 LMH;
(b) the flux decay of the nanofilter is about 0 to about 100%;
(c) the throughput of the nanofilter is about 0 to about 5 kg/m2; and/or
(d) the total yield of the DVD-Ig preparation is about 22 to about 100%.

45. The method of any one of claims 34-44, wherein there is an overall reduction in the total number of viral particles in the DVD-Ig preparation compared to the sample mixture.

46. The method of claim 45, wherein the viral particles in the in the DVD-Ig preparation are selected from the group consisting of XMuLV and MMV.

47. The method of claim 45, wherein the overall reduction in the total number of viral particles in the DVD-Ig preparation is greater than a 3 log reduction value (LRV).

48. A composition comprising a DVD-Ig produced according to the method of claim 34.

49. The composition of claim 48, wherein the composition is a pharmaceutical composition for the treatment of a disease or disorder.

50. The pharmaceutical composition of claim 48, said compositing further comprising a pharmaceutically acceptable carrier.

51. The pharmaceutical composition of claim 50, said compositing further comprising an additional therapeutic agent.

52. The pharmaceutical composition of claim 50, wherein the pharmaceutical composition is administered to an individual, and optionally, wherein the administration is parenteral.

53. A method for producing a product- or process-related impurity reduced DVD-Ig preparation from a load sample mixture comprising a DVD-Ig and at least one product- or process-related impurity said method comprising the steps of:
(a) contacting said load sample mixture to a hydrophobic interaction chromatography resin; and
(b) collecting a product sample, wherein
said product sample comprises said product- or process-related impurity-reduced DVD-Ig preparation.
